(19) United States
(12) Patent Application Publication
(10) Pub. No.: US 2017/0319638 A1
(43) Pub. Date: Nov. 9, 2017

(54) TREATMENT OF CANCER

(71) Applicant: Virttu Biologics Limited, Newhouse (GB)

(72) Inventors: Joe CONNER, Lanarkshire (GB); Timothy CRIPE, Lanarkshire (GB)

(21) Appl. No.: 15/588,207

(22) Filed: May 5, 2017

Related U.S. Application Data
(60) Provisional application No. 62/333,133, filed on May 6, 2016.

(51) Int. Cl. A61K 35/763; A61K 39/245; G01N 33/50; C12N 7/00; A61K 35/76

(52) U.S. Cl. CPC A61K 35/763; G01N 33/5005; C12N 7/00; A61K 39/245; A61K 35/76; C12N 2710/16632

(57) ABSTRACT

A method of treating cancer in a subject is disclosed, the method comprising administration of an oncolytic herpes simplex virus and administration of lymphocyte cells modified to express a chimeric antigen receptor (CAR) or modified to express a T cell receptor (TCR).

### CROSS-REFERENCE TO RELATED APPLICATION

This application claims the benefit of U.S. Provisional Application No. 62/333,133, filed May 6, 2016, the entire disclosure of which is hereby incorporated by reference.

### FIELD OF THE INVENTION

The present invention relates to methods for the treatment of cancer and particularly, although not exclusively, to a method of treating cancer in a subject, the method comprising administration of an oncolytic herpes simplex virus and administration of lymphocyte cells modified to express a chimeric antigen receptor (CAR) or modified to express a T cell receptor (TCR).

### BACKGROUND TO THE INVENTION

Oncolytic virotherapy concerns the use of lytic viruses which selectively infect and kill cancer cells. Some oncolytic viruses are promising therapies as they display exquisite selection for replication in cancer cells and their self-limiting propagation within tumors results in fewer toxic side effects. Several oncolytic viruses have shown great promise in the clinic (Bell, J., Oncolytic Viruses: An Approved Product on the Horizon? Mol Ther. 2010; 18(2): 233-234).

Adoptive transfer of T cells modified to express chimeric antigen receptors (CARs) has had clinical success in B-lymphocyte derived malignancies, however the clinical efficacy of CAR-T cells remains limited in solid tumors (Nishio and Dotti., Oncolmmunology 4:2, e988098; February 2015). WO2014/170389 describes combining oncolytic adenoviral vectors coding for cytokines with adoptive cell therapeutics for cancer treatment.

### SUMMARY OF THE INVENTION

In one aspect of the present invention a method of treating cancer in a subject is provided, the method comprising administration of an oncolytic herpes simplex virus and administration of lymphocyte cells modified to express a chimeric antigen receptor (CAR) or modified to express a T cell receptor (TCR).

In another aspect of the present invention an oncolytic herpes simplex virus for use in a method of treating cancer in a subject is provided, the method comprising administration of an oncolytic herpes simplex virus and administration of lymphocyte cells modified to express a chimeric antigen receptor (CAR) or modified to express a T cell receptor (TCR).

In another aspect of the present invention lymphocyte cells modified to express a chimeric antigen receptor (CAR) or modified to express a T cell receptor (TCR) for use in a method of treating cancer in a subject is provided, the method comprising administration of an oncolytic herpes simplex virus and administration of lymphocyte cells modified to express a chimeric antigen receptor (CAR) or modified to express a T cell receptor (TCR).

In another aspect of the present invention the use of an oncolytic herpes simplex virus in the manufacture of a medicament for use in a method of treating cancer in a subject is provided, the method comprising administration of an oncolytic herpes simplex virus and administration of lymphocyte cells modified to express a chimeric antigen receptor (CAR) or modified to express a T cell receptor (TCR).

In another aspect of the present invention the use of lymphocyte cells modified to express a chimeric antigen receptor (CAR) or modified to express a T cell receptor (TCR) in the manufacture of a medicament for use in a method of treating cancer in a subject is provided, the method comprising administration of an oncolytic herpes simplex virus and administration of lymphocyte cells modified to express a chimeric antigen receptor (CAR) or modified to express a T cell receptor (TCR).

In some preferred embodiments the lymphocyte cells are T-cells, for example cytotoxic T-cells, CD8+ T cells or CD4+ T cells. In some embodiments the lymphocyte cells are natural killer (NK) cells.

The cancer may preferably be a solid tumor.

The CAR or TCR may target an antigen selected from the group consisting of GD2, CD44v7/8, DNAM-1 (DNAX accessory molecule-1), EGP-40 (epithelial glycoprotein-40), EpCAM (endothelial cell adhesion molecule), FBP (folate-binding protein), FR, GD3, VEGFR2, LMP-1 (latent membrane protein 1), MUC1 (mucin 1), PSCA (prostate stem cell antigen), α-folate receptor, CD171, CAIX, Her2, IL13Rα2, IL13R, IL3RA, CEA, CD19, CD20, Lewis-Y, CD33, CD38 (also known as cyclic ADP ribose hydrolase), CD123, gp100, MART1, CEA, CAIX, Her2/Neu, MAGE-A3/A19/A12, MAGE-A3/titin, CD19, GD2, NY-ESO-1, CTAG1B, MAGE-A1, MAGE-C1, SSX2, MAGE-A2B, Brachyury, NY-BR1, BCMA, KRAS (e.g. KRAS G13D, KRAS G12V, KRAS G12R, KRAS G12D, KRAS G12C), KIT, PD-L1, EGFRviii, HPV 16 E6, HPV 16 E7, HPV18 E6, HPV18 E7 and other tumor associated antigens.

The CAR may comprise a binding moiety in the form of a single chain variable fragment (scFv) providing specific and high affinity to the target molecule. The CAR may comprise a cytoplasmic signalling domain derived from CD3 zeta. The CAR may comprise a cytoplasmic signalling domain derived from CD28. The CAR may comprise cytoplasmic signalling domains derived from CD28 and CD3 zeta. The CAR may comprise cytoplasmic signalling domains derived from CD28, OX40, and CD3zeta.

In some embodiments administration of the oncolytic herpes simplex virus and lymphocyte cells is simultaneous or sequential.

In some embodiments the oncolytic herpes simplex virus is administered to the blood, for example the oncolytic herpes simplex virus is administered by intravenous infusion or intra-arterial infusion.

In some embodiments the oncolytic herpes simplex virus is administered by intratumoral injection.

In some embodiments the administration of human lymphocyte cells is part of a method of autologous therapy.

In some embodiments the oncolytic herpes simplex virus does not express, or is not (or has not been) modified to express, a cytokine or chemokine.

In some embodiments the oncolytic herpes simplex virus, preferably the oncolytic herpes simplex virus genome, does not contain, or is not (or has not been) modified to contain, nucleic acid encoding at least one copy of a polypeptide that is heterologous to the virus.

In some embodiments the oncolytic herpes simplex virus is an HSV-1 strain 17+ or mutant thereof. In some embodiments the oncolytic herpes simplex virus is HSV1716.

In another aspect of the present invention a method of increasing the efficacy of adoptive lymphocyte cell therapy in a subject by administering an oncolytic herpes simplex virus to a subject in need thereof is provided. In another aspect of the present invention an oncolytic herpes simplex for use in a method of increasing the efficacy of adoptive cell therapy in a subject is provided, the method comprising administering an oncolytic herpes simplex virus to a subject in need thereof. In another aspect of the present invention the use of an oncolytic herpes simplex virus in a method of increasing the efficacy of adoptive cell therapy in a subject is provided, the method comprising administering an oncolytic herpes simplex virus to a subject in need thereof. The method may comprise increasing the efficacy of anti-tumor response. The method may comprise administration of lymphocyte cells before, during or after administration of oncolytic herpes simplex virus. Such administration of oncolytic herpes simplex virus and lymphocyte cells may be simultaneous or sequential.

In another aspect of the present invention a kit is provided comprising at least one container having a predetermined quantity of oncolytic herpes simplex virus, and at least one container having a predetermined quantity of lymphocytes modified to express a chimeric antigen receptors (CAR) or T cell receptor (TCR).

In some embodiments the oncolytic herpes simplex virus and lymphocytes are provided in separate containers. In other embodiments the kit comprises a container having a mixture of a predetermined quantity of oncolytic herpes simplex virus and predetermined quantity of lymphocytes.

The following numbered paragraphs contain statements of broad combinations of the inventive technical features herein disclosed:—

1. An oncolytic herpes simplex virus for use in a method of treating cancer in a subject, the method comprising administration of an oncolytic herpes simplex virus and administration of lymphocyte cells modified to express a chimeric antigen receptor (CAR) or modified to express a T cell receptor (TCR).

2. Lymphocyte cells modified to express a chimeric antigen receptor (CAR) or modified to express a T cell receptor (TCR) for use in a method of treating cancer in a subject, the method comprising administration of an oncolytic herpes simplex virus and administration of lymphocyte cells modified to express a chimeric antigen receptor (CAR) or modified to express a T cell receptor (TCR).

3. The oncolytic herpes simplex virus or lymphocyte cells for use in a method of treating cancer in a subject of paragraph 1 or 2, wherein the lymphocyte cells are T-cells.

4. The oncolytic herpes simplex virus or lymphocyte cells for use in a method of treating cancer in a subject of any one of paragraphs 1 to 3, wherein the T-cells are cytotoxic T-cells, CD8+ T cells or CD4+ T cells.

5. The oncolytic herpes simplex virus or lymphocyte cells for use in a method of treating cancer in a subject of any one of paragraphs 1 to 3, wherein the lymphocyte cells are NK cells.

6. The oncolytic herpes simplex virus or lymphocyte cells for use in a method of treating cancer in a subject of any one of paragraphs 1 to 5, wherein the cancer is a solid tumor.

7. The oncolytic herpes simplex virus or lymphocyte cells for use in a method of treating cancer in a subject of any one of paragraphs 1 to 6, wherein the CAR or TCR targets an antigen selected from the group consisting of GD2, CD44v7/8, DNAM-1 (DNAX accessory molecule-1), EGP-40 (epithelial glycoprotein-40), EpCAM (endothelial cell adhesion molecule), FBP (folate-binding protein), FR, GD3, VEGFR2, LMP-1 (latent membrane protein 1), MUC1 (mucin 1), PSCA (prostate stem cell antigen), α-folate receptor, CD171, CAIX, Her2, IL13Rα2, IL13R, IL3RA, CEA, CD19, CD20, Lewis-Y, CD33, CD38 (also known as cyclic ADP ribose hydrolase), CD123, gp100, MART1, CEA, CAIX, Her2//Neu, MAGE-A3/A19/A12, MAGE-A3/titin, CD19, GD2, NY-ESO-1, CTAG1B, MAGE-A1, MAGE-C1, SSX2, MAGE-A2B, Brachyury, NY-BR1, BCMA, KRAS (e.g. KRAS G13D, KRAS G12V, KRAS G12R, KRAS G12D, KRAS G12C), KIT, PD-L1, EGFRviii, HPV 16 E6, HPV 16 E7, HPV18 E6, HPV18 E7 and other tumor associated antigens

8. The oncolytic herpes simplex virus or lymphocyte cells for use in a method of treating cancer in a subject of any one of paragraphs 1 to 7, wherein the administration of the oncolytic herpes simplex virus and lymphocyte cells is simultaneous or sequential.

9. The oncolytic herpes simplex virus or lymphocyte cells for use in a method of treating cancer in a subject of any one of paragraphs 1 to 8, wherein the oncolytic herpes simplex virus is administered to the blood.

10. The oncolytic herpes simplex virus or lymphocyte cells for use in a method of treating cancer in a subject of any one of paragraphs 1 to 8, wherein the oncolytic herpes simplex virus is administered by intratumoral injection.

11. The oncolytic herpes simplex virus or lymphocyte cells for use in a method of treating cancer in a subject of any one of paragraphs 1 to 10, wherein the oncolytic herpes simplex virus does not express, or is not modified to express, a cytokine or chemokine.

12. The oncolytic herpes simplex virus or lymphocyte cells for use in a method of treating cancer in a subject of any one of paragraphs 1 to 11, wherein the oncolytic herpes simplex virus does not contain, or is not modified to contain, nucleic acid encoding at least one copy of a polypeptide that is heterologous to the virus.

13. The oncolytic herpes simplex virus or lymphocyte cells for use in a method of treating cancer in a subject of any one of paragraphs 1 to 12, wherein the oncolytic herpes simplex virus is an HSV-1 strain 17+ or mutant thereof.

14. The oncolytic herpes simplex virus or lymphocyte cells for use in a method of treating cancer in a subject of any one of paragraphs 1 to 13, wherein the oncolytic HSV is HSV1716.

15. An oncolytic herpes simplex for use in a method of increasing the efficacy of adoptive cell therapy in a subject, the method comprising administering an oncolytic herpes simplex virus to a subject in need thereof.

16. A kit comprising at least one container having a predetermined quantity of oncolytic herpes simplex virus, and at least one container having a predetermined quantity of lymphocytes modified to express a chimeric antigen receptor (CAR) or T cell receptor (TCR).

17. The kit of paragraph 16, wherein the oncolytic herpes simplex virus and lymphocytes are in separate containers.

18. The kit of paragraph 17, wherein the kit comprises a container having a mixture of a predetermined quantity of oncolytic herpes simplex virus and predetermined quantity of lymphocytes.

### DESCRIPTION

The present invention concerns co-therapy for the treatment of cancer in which a subject receives adoptive transfer of lymphocyte cells modified to express chimeric antigen receptors (CARs) or specifically select T cell receptors (TCRs) and administration of an oncolytic herpes simplex virus.

The inventors have identified that combined administration of lymphocytes modified to express a CAR or TCR and an oncolytic herpes simplex virus enhances persistence CAR/TCR induced response in models of solid tumor, noting that such persistence is present even in tumor cells not susceptible to lysis by the oncolytic herpes simplex virus.

Modified lymphocytes displayed increased migration toward oncolytic HSV-infected tumor cells over non-infected cells and mice treated with combination therapy had significantly delayed tumor growth and prolonged survival when compared to lymphocyte treatment alone. Despite being athymic nude mice, the majority of mice cured by combination therapy were resistant to tumor re-challenge, suggesting long-term persistence of CAR T cells.

Oncolytic Herpes Simplex Virus

An oncolytic virus is a virus that will lyse cancer cells (oncolysis), preferably in a preferential or selective manner. Viruses that selectively replicate in dividing cells over non-dividing cells are often oncolytic. Oncolytic viruses are well known in the art and are reviewed in Molecular Therapy Vol. 18 No. 2 Feb. 2010 pg 233-234.

The herpes simplex virus (HSV) genome comprises two covalently linked segments, designated long (L) and short (S). Each segment contains a unique sequence flanked by a pair of inverted terminal repeat sequences. The long repeat (RL or R_{L}) and the short repeat (RS or R_{S}) are distinct.

The HSV ICP34.5 (also called γ34.5) gene, which has been extensively studied, has been sequenced in HSV-1 strains F and syn17+ and in HSV-2 strain HG52. One copy of the ICP34.5 gene is located within each of the RL repeat regions. Mutants inactivating one or both copies of the ICP34.5 gene are known to lack neurovirulence, i.e. be avirulent/non-neurovirulent (non-neurovirulence is defined by the ability to introduce a high titre of virus (approx 10⁶ plaque forming units (pfu)) to an animal or patient without causing a lethal encephalitis such that the LD₅₀ in animals, e.g. mice, or human patients is in the approximate range of ≦10⁶ pfu), and be oncolytic.

Preferred oncolytic Herpes Simplex Virus (oHSV) are replication-competent virus, being replication-competent at least in the target tumor/cancer cells.

Oncolytic HSV that may be used in the present invention include HSV in which one or both of the γ34.5 (also called ICP34.5) genes are modified (e.g. by mutation which may be a deletion, insertion, addition or substitution) such that the respective gene is incapable of expressing, e.g. encoding, a functional ICP34.5 protein. Preferably, in HSV according to the invention both copies of the γ34.5 gene are modified such that the modified HSV is not capable of expressing, e.g. producing, a functional ICP34.5 protein.

In some embodiments the oncolytic herpes simplex virus may be an ICP34.5 null mutant where all copies of the ICP34.5 gene present in the herpes simplex virus genome (two copies are normally present) are disrupted such that the herpes simplex virus is incapable of producing a functional ICP34.5 gene product. In other embodiments the oncolytic herpes simplex virus may lack at least one expressible ICP34.5 gene. In some embodiments the herpes simplex virus may lack only one expressible ICP34.5 gene. In other embodiments the herpes simplex virus may lack both expressible ICP34.5 genes. In still other embodiments each ICP34.5 gene present in the herpes simplex virus may not be expressible. Lack of an expressible ICP34.5 gene means, for example, that expression of the ICP34.5 gene does not result in a functional ICP34.5 gene product.

Oncolytic herpes simplex virus may be derived from any HSV including any laboratory strain or clinical isolate (non-laboratory strain) of HSV. In some preferred embodiments the HSV is a mutant of HSV-1 or HSV-2. Alternatively the HSV may be an intertypic recombinant of HSV-1 and HSV-2. The mutant may be of one of laboratory strains HSV-1 strain 17, HSV-1 strain F or HSV-2 strain HG52. The mutant may be of the non-laboratory strain JS-1. Preferably the mutant is a mutant of HSV-1 strain 17. The herpes simplex virus may be one of HSV-1 strain 17 mutant 1716, HSV-1 strain F mutant R3616, HSV-1 strain F mutant G207, HSV-1 mutant NV1020, or a further mutant thereof in which the HSV genome contains additional mutations and/or one or more heterologous nucleotide sequences. Additional mutations may include disabling mutations, which may affect the virulence of the virus or its ability to replicate. For example, mutations may be made in any one or more of ICP6, ICP0, ICP4, ICP27. Preferably, a mutation in one of these genes (optionally in both copies of the gene where appropriate) leads to an inability (or reduction of the ability) of the HSV to express the corresponding functional polypeptide. By way of example, the additional mutation of the HSV genome may be accomplished by addition, deletion, insertion or substitution of nucleotides.

A number of oncolytic herpes simplex viruses are known in the art. Examples include HSV1716, R3616 (e.g. see Chou & Roizman, Proc. Natl. Acad. Sci. Vol. 89, pp. 3266-3270, April 1992), G207 (Toda et al, Human Gene Therapy 9:2177-2185, Oct. 10, 1995), NV1020 (Geevarghese et al, Human Gene Therapy 2010 September; 21(9):1119-28), RE6 (Thompson et al, Virology 131, 171-179 (1983)), and Oncovex™ (Simpson et al, Cancer Res 2006; 66:(9) 4835-4842 May 1, 2006; Liu et al, Gene Therapy (2003): 10, 292-303), dlsptk, hrR3,R4009, MGH-1, MGH-2, G47Δ, Myb34.5, DF3γ34.5, HF10, NV1042, RAMBO, rQNestin34.5, R5111, R-LM113, CEAICP4, CEAγ34.5, DF3γ34.5, KeM34.5 (Manservigi et al, The Open Virology Journal 2010; 4:123-156), rRp450, M032 (Campadelli-Fiume et al, Rev Med. Virol 2011; 21:213-226), Baco1 (Fu et al, Int. J. Cancer 2011; 129(6):1503-10) and M032 and C134 (Cassady et al, The Open Virology Journal 2010; 4:103-108).

In some preferred embodiments the herpes simplex virus is HSV-1 strain 17 mutant 1716 (HSV1716). HSV 1716 is an oncolytic, non-neurovirulent HSV and is described in EP 0571410, WO 92/13943, Brown et al (Journal of General Virology (1994), 75, 2367-2377) and MacLean et al (Journal of General Virology (1991), 72, 631-639). HSV 1716 has been deposited on 28 Jan. 1992 at the European Collection of Animal Cell Cultures, Vaccine Research and Production Laboratories, Public Health Laboratory Services, Porton Down, Salisbury, Wiltshire, SP4 0JG, United Kingdom under accession number V92012803 in accordance with the provisions of the Budapest Treaty on the International Recognition of the Deposit of Microorganisms for the Purposes of Patent Procedure (herein referred to as the ‘Budapest Treaty’).

In some embodiments the herpes simplex virus is a mutant of HSV-1 strain 17 modified such that both ICP34.5 genes do not express a functional gene product, e.g. by mutation (e.g. insertion, deletion, addition, substitution) of the ICP34.5 gene, but otherwise resembling or substantially resembling the genome of the wild type parent virus HSV-1 strain 17+. That is, the virus may be a variant of HSV1716, having a genome mutated so as to inactivate both copies of the ICP34.5 gene of HSV-1 strain 17+ but not otherwise altered to insert or delete/modify other protein coding sequences.

In some embodiments the genome of an oncolytic Herpes Simplex Virus according to the present invention may be further modified to contain nucleic acid encoding at least one copy of a polypeptide that is heterologous to the virus (i.e. is not normally found in wild type virus) such that the polypeptide can be expressed from the nucleic acid. As such, the oncolytic virus may also be an expression vector from which the polypeptide may be expressed. Examples of such viruses are described in WO2005/049846 and WO2005/049845.

In order to effect expression of the polypeptide, nucleic acid encoding the polypeptide is preferably operably linked to a regulatory sequence, e.g. a promoter, capable of effecting transcription of the nucleic acid encoding the polypeptide. A regulatory sequence (e.g. promoter) that is operably linked to a nucleotide sequence may be located adjacent to that sequence or in close proximity such that the regulatory sequence can effect and/or control expression of a product of the nucleotide sequence. The encoded product of the nucleotide sequence may therefore be expressible from that regulatory sequence.

In some preferred embodiments, the oncolytic Herpes Simplex Virus is not modified to contain nucleic acid encoding at least one copy of a polypeptide (or other nucleic acid encoded product) that is heterologous to the virus. That is the virus is not an expression vector from which a heterologous polypeptide or other nucleic acid encoded product may be expressed. Such oHSV are not suitable for, or useful in, gene therapy methods and the method of medical treatment for which they are employed may optionally be one that does not involve gene therapy.

In some embodiments the genome of an oncolytic Herpes Simplex Virus according to the present invention does not encode (or is not further modified to contain nucleic acid encoding) a cytokine or chemokine, e.g. a mammalian or human cytokine or chemokine. For example, the genome of an oncolytic Herpes Simplex Virus according to the present invention does not encode an interleukin, e.g. IL-2, a member of the CC family, e.g. CCL5, a member of the CXC family or a member of the CXC family.

In some embodiments the oncolytic herpes simplex virus has an intact ICP0 gene, capable of expressing functional ICP0. In some embodiments the oncolytic herpes simplex virus has an intact ICP27 gene, capable of expressing functional ICP27. In some embodiments the oncolytic herpes simplex virus has an intact vhs gene, capable of expressing functional vhs.

In some embodiments the oncolytic herpes simplex virus has an intact ICP47 gene, capable of expressing functional ICP47.

Optionally, in some embodiments the oncolytic herpes simplex virus does not encode or express (granulocyte macrophage colony stimulating factor) GMCSF.

Optionally, in some embodiments the oncolytic herpes simplex virus is not a herpes simplex virus that lacks functional ICP34.5 genes and lacks a functional ICP47 gene and comprises a gene encoding human GM-CSF.

In some optional embodiments the oncolytic herpes simplex virus is not Talimogene laherparepvec, HSV-1 [strain JS1] ICP34.5-/ICP47-/hGM-CSF also known as OncoVEX GM-CSF (Lui et al., Gene Therapy, 10:292-303, 2003; U.S. Pat. No. 7,223,593 and U.S. Pat. No. 7,537,924)]. In talimogene laherparepvec, the HSV-1 viral genes encoding ICP34.5 are functionally deleted, the ICP47 is functionally deleted, the coding sequence for human GM-CSF is inserted into the viral genome such that it replaces nearly all of the ICP34.5 gene and the HSV thymidine kinase (TK) gene remains intact.

Oncolytic herpes simplex viruses may be formulated as medicaments and pharmaceutical compositions for clinical use and in such formulations may be combined with a pharmaceutically acceptable carrier, diluent or adjuvant. The composition may be formulated for topical, parenteral, systemic, intracavitary, intravenous, intra-arterial, intramuscular, intrathecal, intraocular, intratumoral, subcutaneous, oral or transdermal routes of administration which may include injection. Suitable formulations may comprise the virus in a sterile or isotonic medium. Medicaments and pharmaceutical compositions may be formulated in fluid (including gel) or solid (e.g. tablet) form. Fluid formulations may be formulated for administration by injection or via catheter to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Targeting therapies may be used to deliver the oncolytic virus to certain types of cell, e.g. by the use of targeting systems such as antibody or cell specific ligands. Targeting may be desirable for a variety of reasons; for example if the virus is unacceptably toxic in high dose, or if it would otherwise require too high a dosage, or if it would not otherwise be able to enter the target cells.

HSV capable of targeting cells and tissues are described in (PCT/GB2003/000603; WO 03/068809), hereby incorporated in its entirety by reference.

An oncolytic virus may be administered alone or in combination with other treatments, either simultaneously or sequentially dependent upon the condition to be treated. Such other treatments may include chemotherapy (including either systemic treatment with a chemotherapeutic agent or targeted therapy using small molecule or biological molecule (e.g. antibody) based agents that target key pathways in tumor development, maintenance or progression) or radiotherapy provided to the subject as a standard of care for treatment of the cancer.

In addition to direct action of oncolytic herpes simplex virus (oHSV) on tumors, there is growing evidence that the host immune response plays an important role in establishing the efficacy of the anti-tumor response through innate immune effectors, adaptive antiviral immune responses and adaptive antitumor immune responses (e.g. see Prestwich et al., Oncolytic viruses: a novel form of immunotherapy. Expert Rev Anticancer Ther. October 2008; 8(10): 1581-1588).

Several studies have shown that oHSV is capable of inducing an anti-tumor immune response. This can manifest as tumor growth reduction in lesions treated with oHSV and in untreated lesions in the same animal, efficacy of oHSV requiring an intact immune response, induction of anti-tumor cytokine response, reversal of tumor immune dysfunction and facilitation of tumor antigen presentation. Induction of an anti-tumor immune response can reduce establishment of metastases, or contribute to their elimination, and protect from re-occurrence of tumor.

For example, in Benencia et al., ((2008) Herpes virus oncolytic therapy reverses tumor immune dysfunction and facilitates tumor antigen presentation. Cancer Biol. Ther. 7, 1194-1205) growth reduction in treated and untreated lesions was reported. In Miller and Fraser ((2003) Requirement of an integrated immune response for successful neuroattenuated HSV-1 therapy in an intracranial metastatic melanoma model. Mol. Ther. 7(6):741-747) efficacy of HSV176 required an intact immune response which was mediated by a tumor-specific proliferative T cell response.

Administration of Oncolytic Herpes Simplex Virus

Administration of oncolytic herpes simplex virus is preferably for a period of time sufficient to elicit a treatment effect.

This may involve administration at regular intervals, e.g. weekly or fortnightly, of doses of oncolytic herpes simplex virus sufficient to elicit a treatment effect. For example, doses may be given at regular, defined, intervals over a period of one of at least 1, 2, 3, 4, 5, 6, 7, 8, weeks or 1, 2, 3, 4, 5 or 6 months.

As such, multiple doses of oncolytic herpes simplex virus may be administered. For example, 1, 2, 3, 4, 5, 6, 7, 8, 9, 10 or more doses of oncolytic herpes simplex virus may be administered to a subject as part of a course of treatment. In some preferred embodiments one of at least 2, 3, or 4 doses of oncolytic herpes simplex virus are administered to the subject, preferably at regular intervals (e.g. weekly).

Doses of oncolytic herpes simplex virus may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days). The dose of oncolytic herpes simplex virus given at each dosing point may be the same, but this is not essential. For example, it may be appropriate to give a higher priming dose at the first, second and/or third dosing points.

Suitable dosage amounts of oncolytic herpes simplex virus may be in the range 10⁶ to 10⁹ iu/ml. The term ‘infectious units’ is used to refer to virus concentrations derived using the TCID50 method and ‘plaque forming units (pfus)’ to refer to plaque-based assay results. As 1 iu will form a single plaque in a titration assay, 1 iu is equivalent to 1 pfu.

In general, administration is preferably in a “effective amount”, this being sufficient to elicit a treatment effect in the individual and/or for the virus to have an independent treatment effect on the cancer. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration of oncolytic herpes simplex virus may preferably be carried out for a period of time prior to administration of lymphocyte cells in which period the subject receives oncolytic herpes simplex virus but does not receive lymphocyte cells. This may be referred to as “pre-treatment” with oncolytic herpes simplex virus.

In some preferred embodiments pre-treatment may involve a period of time in which the subject is administered oncolytic herpes simplex virus but is not administered lymphocyte cells (called “oncolytic herpes simplex virus monotherapy” herein). During a period of oncolytic herpes simplex virus monotherapy the subject may also receive treatment in the form of simultaneous, sequential or separate administration of other chemotherapy or radiation therapy, e.g. which may be part of the standard of care for the cancer being treated, but in that time period the patient will not receive a therapeutically effective dose of lymphocyte cells.

As such, methods according to the present invention may comprise administration of an oncolytic herpes simplex virus for a period of time in which the subject receives oncolytic herpes simplex virus but does not receive lymphocyte cells.

At a selected time point the subject may begin treatment with lymphocyte cells. That is, the method may then further comprises the administration of lymphocyte cells to the subject.

Accordingly, at a selected time point the period of pre-treatment may end and the subject may then be administered lymphocyte cells. The subject will preferably continue to be administered oncolytic herpes simplex virus simultaneously, sequentially or separately such that the subject receives co-therapy with lymphocyte cells and oncolytic herpes simplex virus. The subject may also receive, or continue to receive, treatment in the form of simultaneous, sequential or separate administration of other chemotherapy or radiation therapy, e.g. which may be part of the standard of care for the cancer being treated.

For example, pre-treatment may occur for one of at least 1, 2, 3, 4, or 5 weeks in which the subject receives oncolytic herpes simplex virus but does not receive lymphocyte cells. By way of example, a subject may receive oncolytic herpes simplex virus monotherapy in the form of weekly doses of oncolytic herpes simplex virus for one of at least 1, 2, 3, 4, 5, 6, 7, 8, weeks or 1, 2, 3, 4, 5 or 6 months.

In other optional embodiments a subject may receive oncolytic herpes simplex virus monotherapy as described above and may discontinue treatment with oncolytic herpes simplex virus and begin receiving treatment with lymphocyte cells. In such embodiments there may be no day on which a subject is receiving co-therapy, i.e. no day on which an ongoing scheduled programme of treatment with oncolytic herpes simplex virus and lymphocyte cells overlaps.

In other embodiments there may a substantial overlap of treatment with oncolytic herpes simplex virus. In one arrangement, co-therapy with oncolytic herpes simplex virus and lymphocyte cells may commence at the start of treatment, or during a period. In other arrangements a short period of oncolytic herpes simplex virus monotherapy may be provided after which the subject begins to also receive treatment with lymphocyte cells, i.e. co-therapy. During co-therapy the oncolytic herpes simplex virus and lymphocyte cells may be administered on the same day or on different days.

Co-therapy may comprises simultaneous or sequential administration of oncolytic herpes simplex virus and lymphocyte cells.

Simultaneous administration refers to administration of the oncolytic herpes simplex virus and lymphocyte cells together, for example as a pharmaceutical composition containing both agents, or immediately after each other and optionally via the same route of administration, e.g. to the same artery, vein or other blood vessel.

Sequential administration refers to administration of one of the oncolytic herpes simplex virus or lymphocyte cells followed after a given time interval by separate administration of the other agent. It is not required that the two agents are administered by the same route, although this is the case in some embodiments. The time interval may be any time interval.

Whilst simultaneous or sequential administration may be intended such that both the oncolytic herpes simplex virus and lymphocyte cells are delivered to the same tumor or tissue to effect treatment it is not essential for both agents to be present in the tumor or tissue in active form at the same time.

However, in some embodiments of sequential administration the time interval is selected such that the oncolytic herpes simplex virus and lymphocyte cells are expected to be present in the tumor or tissue in active form at the same time, thereby allowing for a combined, additive or synergistic effect of the two agents in treating the tumor or tissue. In such embodiments the time interval selected may be any one of 5 minutes or less, 10 minutes or less, 15 minutes or less, 20 minutes or less, 25 minutes or less, 30 minutes or less, 45 minutes or less, 60 minutes or less, 90 minutes or less, 120 minutes or less, 180 minutes or less, 240 minutes or less, 300 minutes or less, 360 minutes or less, or 720 minutes or less, or 1 day or less, or 2 days or less.

Co-therapy with an oncolytic herpes virus occurs may continue for as long as desired or prescribed. In some embodiments, treatment with oncolytic herpes simplex virus may be discontinued in favour of continued treatment with the lymphocyte cells.

Doses of lymphocyte cells may also be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

During co-therapy the subject may also receive, or continue to receive, treatment in the form of simultaneous, sequential or separate administration of other chemotherapy or radiation therapy, e.g. which may be part of the standard of care for the cancer being treated.

Adoptive Transfer of Lymphocytes

Adoptive transfer of lymphocytes, e.g. T cells, generally refers to a process by which lymphocyte cells are obtained from a subject (e.g. a human patient), typically by drawing a blood sample. The lymphocyte cells are then typically treated or altered in some way, and either returned to the same subject or introduced into a different subject. The treatment is typically aimed at providing a lymphocyte cell population with certain desired characteristics to a subject, or increasing the frequency of lymphocyte cells with such characteristics in that subject. For example, adoptive transfer of virus specific T cells is described in Cobbold et al., (2005) J. Exp. Med. 202: 379-386 and Rooney et al., (1998), Blood 92:1549-1555, hereby incorporated by reference in its entirety.

Genetic targeting of lymphocytes to tumor specific targets may be achieved in two ways. One is transfer of a T-cell receptor with known specificity (TCR therapy) and with matched human leukocyte antigen (HLA) type. The other is modification of cells with artificial molecules such as chimeric antigen receptors (CAR). This approach is not dependent on HLA and is more flexible with regard to targeting molecules. For example, single chain antibodies can be used and CARs can also incorporate costimulatory domains. However, the targets of CAR cells need to be on the membrane of target cells, while TCR modifications can utilize intracellular targets.

The lymphocyte cells may be T cells, B cells or natural killer (NK) cells. In preferred embodiments the lymphocyte cells are T cells. T-cells may be alpha-beta or delta-gamma T cells. In some embodiments, lymphocyte cells may be CD8+, CD4+, or CD3+, cytotoxic T cells regulatory T cells, helper T cells, memory T cells. In preferred embodiments the lymphocyte cells are T cells, optionally cytotoxic T-cells, or CD8+ or CD4+ T cells.

The lymphocyte cells may be non-human mammalian (e.g. murine). Most preferably the lymphocyte cells are human. In some embodiments the lymphocyte cells are obtained from a human subject, modified and/or expanded in vitro and returned to the subject as part of a treatment (autologous therapy).

In the present invention a subject receives co-therapy with an oncolytic herpes simplex virus and adoptive transfer of lymphocytes modified to express a CAR or TCR. The CAR or TCR may be non-native (heterologous) to the lymphocytes.

Accordingly, a subject may be administered an adoptive cell therapeutic composition which may comprise lymphocyte cells which have been modified to express one or more target-specific chimeric antigen receptors or which have been modified to express one or more specifically selected T-cell receptors.

In the present invention, adoptive transfer may be performed with the aim of introducing, or increasing the frequency of, tumor cell reactive lymphocyte cells in a subject.

Accordingly, in one aspect, the present invention provides a method of treating or preventing a disease or disorder in a subject, comprising:
(a) isolating at least one lymphocyte cell, e.g. T cell, from a subject;
(b) modifying the at least one lymphocyte cell to express or comprise a CAR or TCR, and;
(c) administering the modified at least one lymphocyte cell to a subject.

In some embodiments, the subject from which the lymphocyte cell is isolated is the subject administered with the modified lymphocyte cell.

The at least one lymphocyte cell modified according to the present invention can be modified according to methods well known to the skilled person. The modification may comprise nucleic acid transfer for permanent or transient expression the transferred nucleic acid.

Any suitable genetic engineering platform may be used to modify a lymphocyte cell according to the present invention. Suitable methods for modifying a lymphocyte cell include the use of genetic engineering platforms such as gamma retroviral vectors, lentiviral vectors, adenovirus vectors, DNA transfection, transposon-based gene delivery and RNA transfection, for example as described in Maus et al., Annu Rev Immunol (2014) 32:189-225, incorporated by reference hereinabove.

In some embodiments the method may comprise one or more of the following steps: taking a blood sample from a subject; isolating at least one lymphocyte cell, e.g. T cell, from the blood sample; culturing the at least one lymphocyte cell in in vitro or ex vivo cell culture; introducing into the at least one lymphocyte cell a CAR or TCR (or nucleic acid encoding a CAR or TCR), thereby modifying the at least one lymphocyte cell; collecting the at least one lymphocyte cell; mixing the modified lymphocyte cell with an adjuvant, diluent, or carrier; administering the modified lymphocyte cell to a subject.

The skilled person is able to determine appropriate reagents and procedures for adoptive transfer of lymphocyte cells according to the present invention for example by reference to Qasim et al., Journal of Hepatology (2015) 62: 486-491, which is incorporated by reference in its entirety.

In one aspect, the methods according to the present invention may comprise:
(a) isolating at least one lymphocyte cell, e.g. T cell, from a subject;
(b) introducing into the at least one lymphocyte cell an isolated nucleic acid or vector encoding a CAR or TCR, thereby modifying the at least one lymphocyte cell; and
(c) administering the modified at least one lymphocyte cell to the subject.

In embodiments according to the present invention the subject is preferably a human subject. In some embodiments, the subject to be treated according to a therapeutic or prophylactic method of the invention herein is selected based on HLA genotype. In some embodiments, the subject has an HLA allele encoding an MHC class I α-chain which in the context of an MHC class I molecule is capable of presenting an tumor associated antigen peptide as described herein. Prophylactic intervention may comprise vaccination, e.g. as described in Weekly epidemiological record 40(84): 405-420 (2009).

T Cell Receptors

T Cell Receptors (TCRs) are heterodimeric, antigen-binding molecules typically comprising an α-chain and a β-chain. In nature, α-chain and a β-chains are expressed at the cell surface of T cells (αβ T cells) as a complex with invariant CD3 chains. An alternative TCR comprising γ and δ chains is expressed on a subset of T cells (γδ T cells). TCRs recognise (bind to) antigen peptide presented by major histocompatibility complex (MHC) molecules. TCR structure and recognition of the peptide-MHC complex is described in detail for example in Immunobiology, 5^{th} Edn. Janeway C A Jr, Travers P, Walport M, et al. New York: Garland Science (2001), Chapters 3 and 6, which are hereby incorporated by reference in their entirety.

TCR α-chain and β-chains comprise a constant (C) region, and a variable (V) region. The variable regions of the α-chain and β-chain polypeptides bind to the antigen-MHC complex. Each TCR α-chain and β-chain variable region comprises three complementary determining regions (CDRs), which determine specificity for the antigen-MHC complex. The CDRs for the TCR α-chain and β-chain are respectively designated CDR1-3a and CDR1-3b. CDR3 of the α-chain and β-chain polypeptides are thought to be the most important CDRs for antigen recognition. The variable regions of the α-chain and β-chain also comprise framework regions between the CDRs.

Engineering of TCRs into T cells may be performed during culture, in vitro, for transduction and expansion, such as happens during expansion of T cells for adoptive T cell therapy. The transduction may utilize a variety of methods, but stable gene transfer is required to enable sustained TCR expression in clonally expanding and persisting T cells. TCRs may recognise both cell surface and intracellular proteins and therefore have some advantages of CARs in some circumstances.

TCR molecules may be designed or identified to target any desired target molecule or cell-surface molecule, e.g. ligand, antigen, cell-surface antigen, receptor or cell-surface receptor. Target molecules may be selected from IL13R, IL3RA, CD38 (also known as cyclic ADP ribose hydrolase), CD123, KIT, PD-L1, gp100, MART1, CEA, CAIX, Her2.Neu, MAGE-A3/A19/A12, MAGE-A3/titin, CD19, GD2, NY-ESO-1, CTAG1B, MAGE-A1, MAGE-C1, SSX2, MAGE-A2B, Brachyury, NY-BR1, BCMA, KRAS (e.g. KRAS G13D, KRAS G12V, KRAS G12R, KRAS G12D, KRAS G12C), EGFRviii, HPV 16 E6, HPV 16 E7, HPV18 E6, HPV18 E7 and other tumor associated antigens (e.g. see Hinrichs C S, Restifo N P. Reassessing target antigens for adoptive T cell therapy. Nature biotechnology. 2013; 31(11):999-1008. doi:10.1038/nbt.2725.).

Chimeric Antigen Receptors

Chimeric Antigen Receptors (CARs) are recombinant receptors that provide both antigen-binding and T cell activating functions. CARs may be combined with costimulatory ligands, chimeric costimulatory receptors or cytokines to further enhance T cell potency, specificity and safety (Sadelain et al., The basic principles of chimeric antigen receptor (CAR) design. Cancer Discov. 2013 April; 3(4): 388-398. doi:10.1158/2159-8290.CD-12-0548, specifically incorporated herein by reference). CARs are recombinant receptors for antigen, typically cell-surface antigens, which, in a single molecule, redirect the specificity and function of T lymphocytes and other immune cells. In cancer immunotherapy they may be used to rapidly generate tumor-targeted T cells.

Engineering of CARs into T cells may be performed during culture, in vitro, for transduction and expansion, such as happens during expansion of T cells for adoptive T cell therapy. The transduction may utilize a variety of methods, but stable gene transfer is required to enable sustained CAR expression in clonally expanding and persisting T cells. CARs may provide a broader range of functional effects than transduced T cell receptors, where strength of signaling, which is for the most part determined by the TCR's affinity for antigen, is the principal determinant of T cell fate (Sadelain et al., supra). However, TCR may recognise both cell surface and intracellular proteins and therefore have some advantages of CARs in some circumstances.

CAR molecules may be designed to target any desired target molecule or cell-surface molecule, e.g. ligand, antigen, cell-surface antigen, receptor or cell-surface receptor. Target molecules may be selected from target associated with solid tumors, such as GD2, CD44v7/8, DNAM-1 (DNAX accessory molecule-1), EGP-40 (epithelial glycoprotein-40), EpCAM (endothelial cell adhesion molecule), FBP (folate-binding protein), FR, GD3, VEGFR2, LMP-1 (latent membrane protein 1), MUC1 (mucin 1), PSCA (prostate stem cell antigen), α-folate receptor, CD171, CAIX, Her2, ID 3Rα2, CEA, and target associated with non-solid tumors (e.g. hematologic malignancy) such as CD19, CD20, Lewis-Y, CD33, IL13R, IL3RA, CD38 (also known as cyclic ADP ribose hydrolase), CD123, KIT, PD-L1. CAR modified T-cells and targets for use in treatment of solid tumors are described by Guo et al (Chimeric Antigen Receptor-Modified T Cells for Solid Tumors: Challenges and Prospects. Journal of Immunology Research Volume 2016 (2016), Article ID 3850839, 11 pages) and by Dai et al (Chimeric Antigen Receptors Modified T-Cells for Cancer Therapy. JNCI J Natl Cancer Inst (2016) 108 (7): djv439 doi: 10.1093/jnci/djv439), both incorporated herein by reference.

CAR molecules may be further engineered to express co-stimulatory endodomains such as those derived from CD28 and tumor necrosis factor receptor superfamily member 9 (TNFRSF9; 4-1BB) to promote T cell proliferation and persistence upon encountering tumor cells (Nishio and Dotti., Oncolmmunology 4:2, e988098; February 2015).

A CAR typically combines an antigen recognition domain of a specific antibody with an intracellular domain of the CD3-zeta chain or FcyRI protein in a single chimeric protein. The structural features of a CAR are described by Sjouke et al., (The pharmacology of second-generation chimeric antigen receptors. Nature Reviews Drug Discovery, 14, 499 509 (2015) doi:10.1038/nrd4597). A CAR typically has an extracellular binding moiety linked to a transmembrane domain and endodomain. An optional hinge or spacer domain may provide separation between the binding moiety and transmembrane domain and may act as a flexible linker.

The binding moiety provides an antigen recognition function and may be derived from an antibody binding domain, e.g. containing the CDR sequences of an antibody to the selected target. The binding moiety may be an scFV. The binding moiety may enable HLA independent antigen recognition. Binding moieties may also be derived from native T cell receptor alpha and beta chains. In principle, binding domains may be derived from any polypeptide sequence that binds a selected target with high affinity.

Hinge or spacer regions may be flexible domains allowing the binding moiety to orient in different directions. Hinge or spacer regions may be derived from IgG1 or the CH₂CH₃ region of immunoglobulin.

Transmembrane domains may be hydrophobic alpha helix that spans the cell membrane. The transmembrane domain associated with the endodomain is commonly used.

The endodomain is responsible for receptor clustering dimerization after antigen binding and for initiation of signal transduction to the cell. One commonly used transmembrane domain is the CD3-zeta transmembrane and endodomain. Intracellular domains from one or more co-stimulatory protein receptors, such as CD28 4-1BB, OX40, ICOS, may optionally be incorporated into the cytoplasmic tail of the CAR to provide additional co-stimulatory signaling, which may be beneficial in terms of anti-tumor activity.

In one embodiment, a CAR comprises an extracellular domain having an antigen recognition domain, a transmembrane domain, and a cytoplasmic domain. A transmembrane domain that is naturally associated with one of the domains in the CAR may be used or the transmembrane domain can be selected or modified by amino acid substitution to avoid binding of such domains to the transmembrane domains of the same or different surface membrane proteins to minimize interactions with other members of the receptor complex.

The cytoplasmic domain may be designed to comprise the CD28 and/or 4-1BB signaling domain by itself or be combined with any other desired cytoplasmic domain(s). The cytoplasmic domain may be designed to further comprise the signaling domain of CD3-zeta. For example, the cytoplasmic domain of the CAR can include but is not limited to CD3-zeta, 4-1BB and CD28 signaling modules and combinations thereof.

In one embodiment, the CART cells of the invention can be generated by introducing a lenfiviral vector in vitro comprising a desired CAR, for example a CAR comprising anti-CD19, CD8a hinge and transmembrane domain, and human 4-1BB and CD3zeta signaling domains, into the cells. The CAR T cells of the invention are able to replicate in vivo resulting in long-term persistence that can lead to sustained tumor control.

In one embodiment the invention relates to administering a genetically modified T cell expressing a CAR for the treatment of a patient having cancer or at risk of having cancer using lymphocyte infusion. Preferably, autologous lymphocyte infusion is used in the treatment. Autologous PBMCs are collected from a patient in need of treatment and T cells are activated and expanded using the methods described herein and known in the art and then infused back into the patient.

T Cells

T cells may be characterised by reference to surface expression of one or more of: a TCR polypeptide (e.g. α, β, γ or δ chain), a CD3 polypeptide (e.g. γ, δ or ε chain), CD8, and CD4. Surface expression of a given polypeptide can be measured by various methods well known in the art, e.g. by antibody-based methods such as immunohistochemistry, immunocytochemistry, and flow cytometry.

In some embodiments T cells are CD3+. In some embodiments the T cells are CD4+. In some embodiments the T cells are CD8+. In some embodiments, T cells are cytotoxic T cells.

CTLs are capable of effecting cell death in cells infected with a virus by releasing cytotoxic factors including perforin, granzymes, granulysin, and/or by inducing apoptosis of the infected cell by ligating FAS on the infected cell through FASL expressed on the T cell (described for example by Chavez-Galan et al., Cellular and Molecular Immunology (2009) 6(1): 15-25, hereby incorporated by reference in its entirety). Cytotoxicity can be investigated, for example, using any of the methods reviewed in Zaritskaya et al., Expert Rev Vaccines (2011), 9(6):601-616, hereby incorporated by reference in its entirety. One example of an assay for cytotoxicity of a T cell for to a target cell is the ⁵¹Cr release assay, in which target cells are treated with ⁵¹Cr, which they internalize. Lysis of the target cells by T cells results in the release of the radioactive ⁵¹Cr into the cell culture supernatant, which can be detected.

TCRs recognize peptides presented by an MHC molecule, specifically an MHC class I molecule. MHC class I molecules are heterodimers of an α-chain and a δ2-microglobulin. The α-chain has three domains designated α1, α2 and α3. The α1 and α2 domains together form the groove to which the peptide presented by the MHC class I molecule binds, to form the peptide-MHC complex. MHC class I α-chains are polymorphic, and different α-chains are capable of binding and presenting different peptides. In humans MHC class I α-chains are encoded by human leukocyte antigen (HLA) genes.

Antigenic peptides may be tumor associated antigens or viral antigens associated with tumor inducing viruses, e.g. Epstein Barr Virus, Human Papillomavirus. Peptides may be presented by an antigen presenting cell (APC). APCs process polypeptides by the molecular machinery to peptides which then become associated with MHC molecules and presented as peptide-MHC complexes at the cell surface. Different TCRs display different ability to bind to, and therefore different reactivity to, different peptide-MHC complexes.

Viral peptides/polypeptides are processed and presented in complex with MHC Class I molecules. Antigen processing, loading and presentation on MHC is described in detail in, for example, Immunobiology, 5^{th} Edn. Janeway C A Jr, Travers P, Walport M, et al. New York: Garland Science (2001), Chapter 5, hereby incorporated by reference in entirety.

Different kinds of T cells are activated through their TCRs by recognition of MHC-peptide complexes. CD8+ T cells recognize peptide-MHC class I complexes. T cell activation requires binding MHC-peptide complex for which the TCR of the T cell has high affinity in the context of a positive costimulatory signal from APC. The process of T cell activation is well known to the skilled person and described in detail, for example, in Immunobiology, 5^{th} Edn. Janeway C A Jr, Travers P, Walport M, et al. New York: Garland Science (2001), Chapter 8, which is incorporated by reference in its entirety.

APCs may be professional APCs. Professional APCs are specialised for presenting antigen to T cells; they are efficient at processing and presenting peptide-MHC at the cell surface, and express high levels of costimulatory molecules. Professional APCs include dendritic cells (DCs), macrophages, and B cells. Non-professional APCs are other cells capable of presenting MHC-peptide complexes to T cells, in particular MHC Class I-peptide complexes to CD8+ T cells.

Cancer

A cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentume, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

In some embodiments the cancer may be a solid tumor. Solid tumors may, for example, be in bladder, bone, breast, eye, stomach, head and neck, germ cell, kidney, liver, lung, nervous tissue, ovary, pancreas, prostate skin, soft-tissues, adrenal gland, nasopharynx, thyroid, retina, and uterus. Solid tumors may include melanoma, rhabdomyosarcoma, Ewing sarcoma, and neuroblastoma.

The cancer may be a pediatric solid tumor, i.e. solid tumor in a child, for example osteosarcoma, chondroblastoma, chondrosarcoma, Ewing sarcoma, malignant germ cell tumor, Wilms tumor, malignant rhabdoid tumor, hepatoblastoma, hepatocellular carcinoma, neuroblastoma, melanoma, adrenocorticoid carcinoma, nasopharyngeal carcinoma, thyroid carcinoma, retinoblastoma, soft-tissue sarcoma, rhabdomyosarcoma, desmoid tumor, fibrosarcoma, liposarcoma, malignant fibrous histiocytoma, neurofibrosarcoma.

The cancer may be positive for an antigen against which the CAR or TCR is directed, e.g. a tumor associated antigen. The cancer may also be positive for nectin 1. ‘Positive’ cancers may have cancer or tumor cells which express or overexpress the respective antigen. Expression may optionally be at the cell-surface.

Subjects

The subject to be treated may be any animal or human. The subject is preferably mammalian, more preferably human. The subject may be a non-human mammal, but is more preferably human. The subject may be male or female. The subject may be a patient. A subject may have been diagnosed with a cancer, or be suspected of having a cancer.

The subject may be a child, i.e. a human subject of age less than 18 years, or of age less than 16 years, or of age less than 14 years, or of age less than 12 years. The age may be determined at the point of first dose with oncolytic herpes simplex virus.

Other Chemotherapeutic Agents

In addition to treating a cancer by using an oncolytic herpes simplex virus with or without lymphocyte cells, subjects being treated may also receive treatment with other chemotherapeutic agents. For example, other chemotherapeutic agents may be selected from:
(i) alkylating agents such as cisplatin, carboplatin, mechlorethamine, cyclophosphamide, chlorambucil, ifosfamide;
(ii) purine or pyrimidine anti-metabolites such as azathiopurine or mercaptopurine;
(iii) alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel;
(iv) topoisomerase inhibitors such as the type I topoisomerase inhibitors camptothecins irinotecan and topotecan, or the type II topoisomerase inhibitors amsacrine, etoposide, etoposide phosphate, teniposide;
(v) antitumor antibiotics (e.g. anthracyline antibiotics) such as dactinomycin, doxorubicin (Adriamycin™), epirubicin, bleomycin, rapamycin;
(vi) antibody based agents, such as anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab,
(vii) EGFR inhibitors such as erlotinib, cetuximab and gefitinib
(viii) anti-angiogenic agents such as bevacizumab (Avastin®).

Routes of Administration

Viruses, lymphocyte cells, chemotherapeutic agents, medicaments and pharmaceutical compositions according to aspects of the present invention may be formulated for administration by a number of routes, including but not limited to, parenteral, intravenous, intra-arterial, intramuscular, intratumoral and oral. Viruses, lymphocyte cells, chemotherapeutic agents, medicaments and pharmaceutical compositions may be formulated in fluid or solid form. Fluid formulations may be formulated for administration by injection to a selected region of the human or animal body.

Kits

In some aspects of the present invention a kit of parts is provided. In some embodiments the kit may have at least one container having a predetermined quantity of oncolytic herpes simplex virus, e.g. predetermined viral dose or number/quantity/concentration of viral particles. The oncolytic herpes simplex virus may be formulated so as to be suitable for injection or infusion to a tumor or to the blood. In some embodiments the kit may further comprise at least one container having a predetermined quantity of lymphocytes modified to express a chimeric antigen receptors (CAR) or T cell receptor (TCR). The lymphocytes may also be formulated so as to be suitable for injection or infusion to the tumor or to the blood. In some embodiments a container having a mixture of a predetermined quantity of oncolytic herpes simplex virus and predetermined quantity of lymphocytes is provided, which may optionally be formulated so as to be suitable for injection or infusion to the tumor or to the blood.

In some embodiments the kit may also contain apparatus suitable to administer one or more doses of the oncolytic herpes simplex virus and/or lymphocytes. Such apparatus may include one or more of a catheter and/or needle and/or syringe, such apparatus preferably being provided in sterile form.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Embodiments and experiments illustrating the principles of the invention will now be discussed with reference to the accompanying figures in which:

FIGS. 1A and 1B. Most mouse tumor cell lines do not endogenously express GD2. FIG. 1A Mouse Fibroblasts (3T3), Neuroblastoma (DFCI-331), Melanoma (B16), and RMS (HGF-116, 76-9, and M3-9-M) Cells were stained for cell surface GD2 and analysed by flow cytometry. FIG. 1B Mouse tumor cell lines were modified to express GD2 by either being fused to dorsal root ganglion cells (NXS2) or by the over-expression of GD2 and GD3 synthases (B78D14, B16/GD2, HGF-116/GD2, and 76-9/GD2) then stained for cell surface GD2 and analysed by flow cytometry.

FIG. 2. Soluble 14G2a scFv binds specifically to GD2-expressing Tumor cells. Mouse cell lines were incubated with soluble 14G2a scFv fused to rat CH₂CH₃. Binding of soluble 14G2a was detected using anti-rat F(ab′)2 and analysed by flow cytometry.

FIGS. 3A-3D. GD2-28z CAR T cells are functionally active in the presence of antigen positive targets. FIG. 3A Time line for generating GD2-28z. FIG. 3B Day 4 T GD2-28z CAR T cells were stained for CD4 and GD2 CAR and analysed by flow cytometry. FIG. 3C Tumor Cells were labelled with Chromium-51 for 1-2 Hours then co-cultured with GD2-28z or mock Transduced T cells for 6 hrs and Chromium-51 release was measured using a scintillation counter. FIG. 3D GD2-28z or Mock transduced T cells were co-cultured in the presence or absence of tumor cells for 24 hrs at a 1:1 ratio and secreted cytokines were measured using CBA beads.

FIG. 4. GD2-28z CAR T Cells persist in vivo and delay tumor growth.

Mice Received 2×10⁶ B78D14 Cells on day 0, 500cGy TBI on day 6, and 4×10⁶ GD2-28z CAR T cells or mock T cells on day 7. CD8+CAR Persistence (A), tumor size (B), and percent survival (C) was measured.

FIG. 5. RMS cell lines express HSV1716 entry receptors and are susceptible to oncolytic lysis. A) RNA Was extracted from three RMS cell lines (M3-9-M, 76-9, And HGF-116GL) and two melanoma lines (B16 and B78D14) And Nectin 1 Expression levels were determined via real-time PCR. (B) Mouse cell lines were infected with HSV1716 at MOI: 0.01, 0.1, 1.0, 10, And 100. Percent survival by MTS assay is shown relative to mock-infected control 2 days post-infection.

FIG. 6. HSV1716 Increases GD2 CAR Persistence in melanoma models. Mice received 500cGy TBI on day 0, 2×10⁶ B78D14 (A) or B16/GD2 (B) Cells on day 1, 500cGy TBI on day 6, and 3×10⁶ GD2-28z CAR T cells or mock T cells on day 7. HSV1716 was administered on days 3, 5, 7, 10, and 13. CAR persistence, tumor size, and percent survival was measured weekly.

FIGS. 7A-7D. HSV1716 and T cells synergize to delay tumor growth and enhance T cell persistence. Mice received 500cGy TBI On day 0, 2×10⁶ 76-9GL/GD2 cells on day 1, and 5×10⁶ GD2-28z CAR T cells or mock T cells on day 7. HSV1716 was administered on days 3, 5, 7, 10, and 13. Adoptively Transferred T cell persistence FIG. 7A, CAR persistence FIG. 7B, tumor size FIG. 7C, and percent survival FIG. 7D were measured over time.

FIG. 8. Cytokine and chemokine profiles of tumor models in response to oHSV infection in vitro. Human Ewing sarcoma model A673 and human neuroblastoma models SK-N-AS and SK-N-BE(2) were cultured with oHSV at a multiplicity of infection (MOI) of 10 and gene expression analysis was performed at 12 hours post infection by RT-PCR.

FIG. 9. Baseline GD2 expression in human Ewing sarcoma xenograft model A673 and human neuroblastoma xenograft models SK-N-AS and SK-N-BE(2) in vitro by flow cytometry analysis. Results represent averages of 3 replicates, each with triplicate samples.

FIG. 10. Migration of human GD2-directed CAR-T cells toward human Ewing sarcoma A673 and neuroblastomas SK-N-AS and SK-N-BE(2) with and without oHSV infection by transwell assay. Human Ewing sarcoma A673 and human neuroblastoma SK-N-AS and SK-N-BE(2) cells were cultured with Seprehvir at a multiplicity of infection (MOI) of 1 for 24 hours and red fluorescent PKH23-stained GD2-directed human CAR-T cells were added into 5 um pore transwell inserts above the cell culture at an E:T ratio of 2:3 for 2 hours. Media alone served as a negative control, while media containing 75 ng/ml CXCL-10 (IP-10) and media containing 10 ng/ml CCL-5 (RANTES) served as positive controls. Cells were quantified through microscopic visualization. Results represent averages of 5 replicates for each sample.

FIG. 11. Survival curve of Ewing sarcoma tumor-bearing mice treated with human GD2-directed CAR-T cells with and without Seprehvir. Athymic nude mice were subcutaneously inoculated with human Ewing sarcoma xenograft A673 cells and tumors were allowed to reach volumes of 200-250 mm³. Mice were treated with either PBS or Seprehvir at a dose of 1e7 PFU intra-tumorally on days 0, 3, and 5. Mice received either PBS or 2.5 mg cyclophosphamide (CPM) intraperitoneally on day 3 as lymphodepletion. On day 6, 1.2e7 total GD2-directed human CAR T-cells (83% CAR positivity) were injected intravenously. Tumor growth and overall survival were monitored for 85 days after the initial treatment.

FIG. 12. No significant change in GD2 expression with oHSV1716 infection in hNBL cell lines SK-N-AS and SK-N-BE2, but increased GD2 expression with infection in hEWS cell line A673. No significant change in GD2 expression with oHSV1716 infection in hNBL cell lines SK-N-AS and SK-N-BE2, but increased GD2 expression with infection in hEWS cell line A673

FIG. 13. No significant change in PD-L1 expression with oHSV1716 infection in pediatric solid tumor cell lines. PD-L1 expression before and after HSV1716 infection of human Ewing sarcoma xenograft model A673 and human neuroblastoma xenograft models SK-N-AS and SK-N-BE(2) in vitro. Cells were infected with HSV1716 at multiplicity of infection (MOI) of 0, 0.1, or 1 for 24 hours and then analyzed by flow cytometry. A673 displayed intermediate PD-L1 expression. SK-N-AS displayed high PD-L1 expression. SK-N-BE(2) displayed modest PD-L1 expression. HSV1716 infection did not significantly affect PD-L1 levels in any of the models.

FIG. 14. oHSV1716-Induced Chemokine/Cytokine Gene Expression in Pediatric Solid Tumor Cell Lines. Human Ewing sarcoma model A673 and human neuroblastoma models SK-N-AS and SK-N-BE(2) were cultured with HSV1716 at MOI 10 and gene expression analysis was performed at 6 hours and 12 hours post infection by RT-PCR. These data suggest that oHSV infection will increase T-cell migration to the tumor site and increase T-cell activation.

FIG. 15. oHSV1716-Induced Chemokine/Cytokine Gene Expression in hNBLSK-N-BE2 In Vivo. Athymic nude mice were implanted with SK-N-BE(2) and treated with 1×107PFU HSV1716 or PBS as control intra-tumorally on days 0, 2, and 4. Data represent average of all samples (n=2 tumors per treatment group). These data suggest that oHSV infection will increase T-cell migration to the tumor site, proliferation and activation. These data also suggest that oHSV1716 induces expression of T-cell inhibitory ligands, and that the addition of a PD-1 inhibitor to oHSV1716 may be of benefit. Human data shown in left bar with mouse data in adjacent right bar.

FIG. 16. Flow scheme for transwell assays.

FIG. 17. Negative controls.

FIG. 18. Positive controls.

FIG. 19. SKNAS hNBL cells Negative Controls: No CARs.

FIG. 20. SKNAS hNBL cells.

FIG. 21. SKNBE2 hNBL cells Negative Control: No CARs.

FIG. 22. SKNBE2 hNBL cells.

FIG. 23. Transwell summary. SK-N-BE2 induces more GD2 hCAR-T cell migration than SK-N-AS at baseline. oHSV1716 infection induces increased migration of GD2 hCAR-T cells toward hNBLcell lines SK-N-AS and SK-N-BE2.

FIG. 24. Historical data.

FIG. 25. Study design: SK-N-AS In Vivo #1 Efficacy of Combination of oHSV1716+GD2 CAR T-cells in Pediatric Solid Tumor Xenograft Model SK-N-AS In Vivo.

FIG. 26. SK-N-AS+/−oHSV+/−CAR-T:Tumor Growth Curves.

FIG. 27. SK-N-AS+/−oHSV+/−CAR-T:Tumor Growth Curves. In the right panel, the line extending furthest right is oHSV+CAR T.

FIG. 28. SK-N-AS+/−oHSV1716+/−CAR-T: Survival Curves. In the right panel, the line extending furthest right is oHSV+CAR T.

FIG. 29. Study design: A673 In Vivo Lymphodepletion Pilot #1 Efficacy of Combination of oHSV1716+GD2 CAR T-cells with and without Lymphodepletion in Pediatric Solid Tumor Xenograft Model A673 In Vivo.

FIG. 30. Charts showing tumor volume in A673+CAR-T cells.

FIG. 31. Summary of methods for Example 2. *pfu=plaque forming unit.

FIG. 32. In vitro results for example 3: Seprehvir infection induces T-cell attractant chemokines and T-cell activating cytokines in vitro.

FIG. 33. In vitro results for example 3: CAR T-cells express chemokine receptors (left). Tumor cells variably express GD2 and PD-L1 (right).

FIG. 34. In vitro results for example 3: Migration of CAR T cells toward SK-N-AS with and without oHSV infection.

FIG. 35. In vivo results for Example 3: Seprehvir significantly delays tumor growth and enhances anti-tumor efficacy of GD2-directed human 3^{rd} generation CAR T-cells against the neuroblastoma xenograft model SK-N-AS.

FIG. 36: In vivo results for Example 3: Seprehvir significantly delays tumor growth and enhances anti-tumor efficacy of human GD2-directed CAR T-cells against human Ewing sarcoma xenograft model A673. Cured mice were re-challenged with tumor cells after 100 days survival.

### EXAMPLES

### Example 1—Evaluation of Attenuated HSV1716 in Combination with Chimeric Antigen Receptor T Cells for Solid Tumors

Neuroblastoma, osteosarcoma, and rhabdomyosarcoma are among the most prevalent childhood solid tumors. Each of these tumor types as well as melanomas exhibit increased levels of the tumor associated carbohydrate, GD2 on their cell surface making them ideal targets for chimeric antigen receptor (CAR) T cell-directed therapies. Despite the ability of GD2 CAR T Cells to target GD2-expressing tumor cells in vitro, there is great interest in improving tumor clearance in vivo, especially for solid tumors where current outcomes remain poor. We hypothesize that the immunosuppressive milieu present within the solid tumor microenvironment serves as a major factor limiting the effectiveness of GD2 CAR T cells and propose that administration of oncolytic viruses could induce inflammation within the tumor microenvironment that may enhance, rather than inhibit, the effectiveness of immune based therapies. GD2 CAR T cells composed of the 14G2a Single chain variable fragment linked to the cytoplasmic signalling domains of CD28 and CD3 zeta (GD2-28z) were expressed in murine lymphocytes and evaluated for the ability to target and lyse GD2-expressing tumor cells. Additionally GD2-28z T cells were co-cultured with tumor cells to access their ability to secrete proinflammatory cytokines IFNγ and IL-2. In order to determine the oncolytic ability of attenuated HSV1716, tumor cells were cultured in the presence or absence of HSV1716 and relative cell survival was measured. We observed specific lysis of GD2-expressing tumor cells when co-cultured with GD2-28z, but not mock T cells. Furthermore, GD2-28z T cells secrete IFNγ and IL-2 following co-culture with GD2-expressing tumor cells. Interestingly, melanoma cell lines were not susceptible to oncolytic lysis while rhabdomyosarcoma (RMS) cell lines were susceptible. Using a melanoma model, GD2-28z T cells displayed anti-tumor activity. The combination of GD2-28z and HSV1716 enhanced CAR persistence in a melanoma model. Given that the melanoma cells in our model are not susceptible to oncolytic lysis yet we observe an increased T cell persistence when used in combination with HSV1716, this supports our hypothesis that HSV1716 in inducing inflammation, which is then triggering T cell expansion.

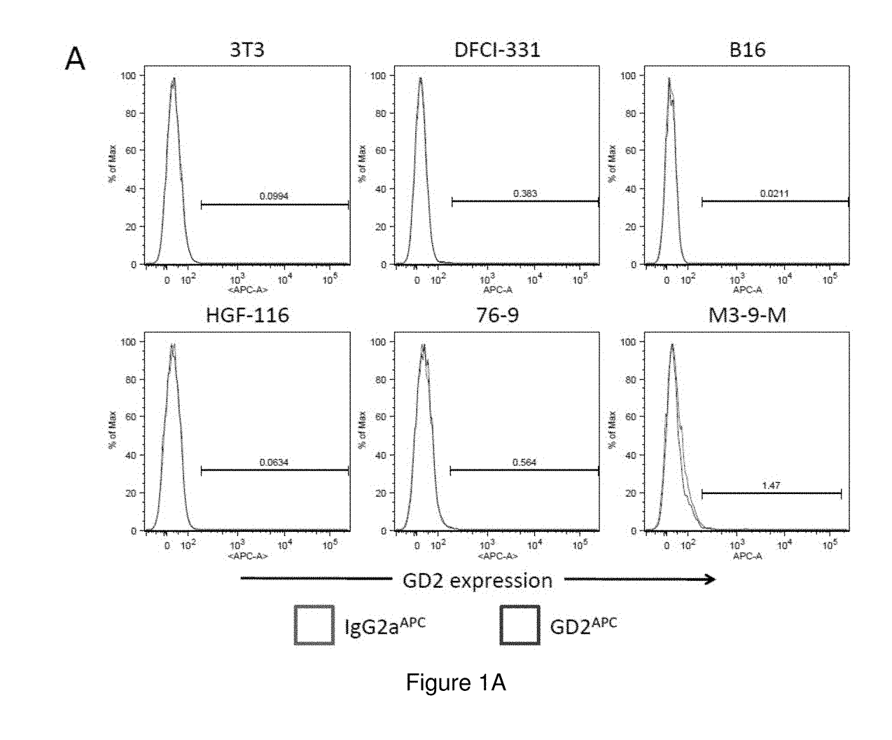

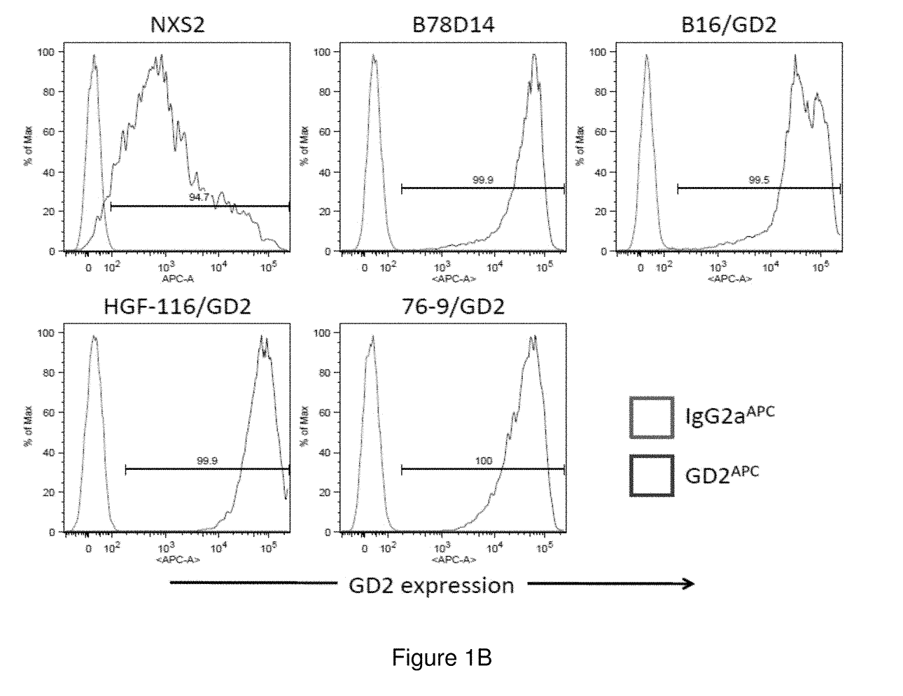

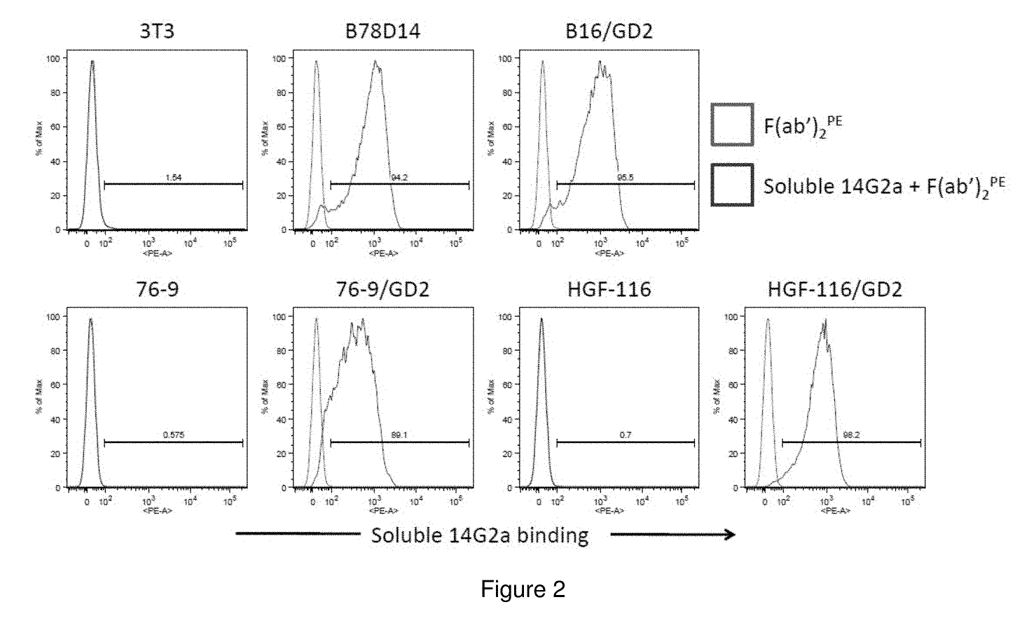

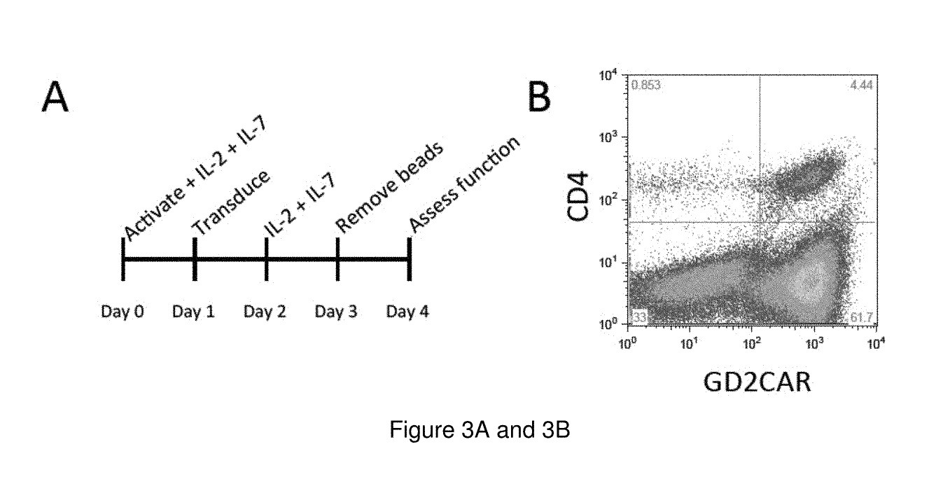

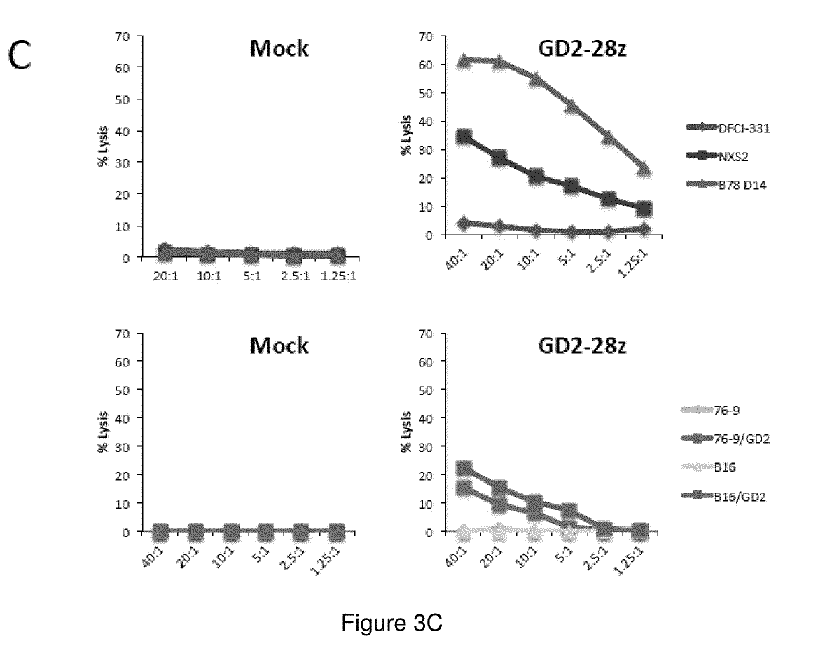

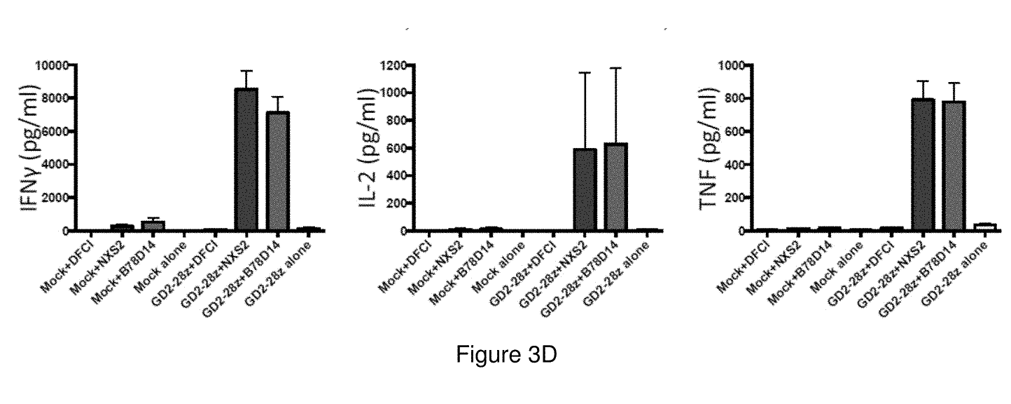

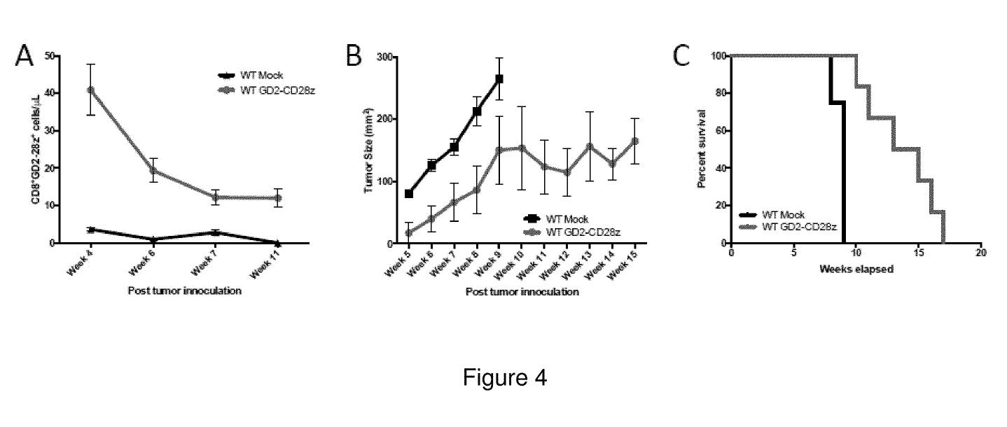

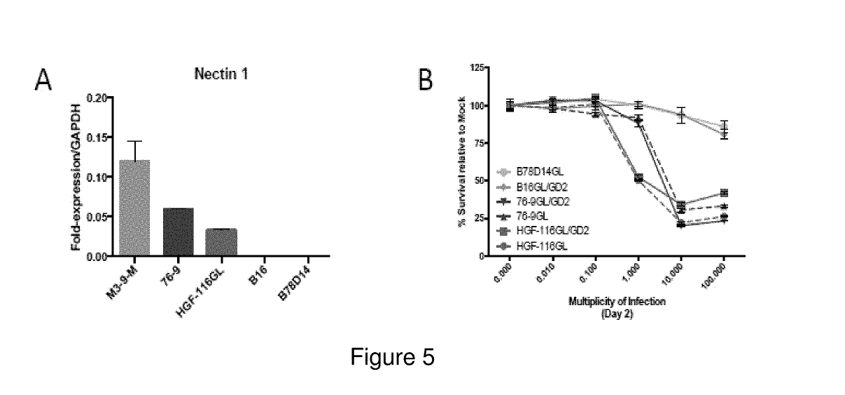

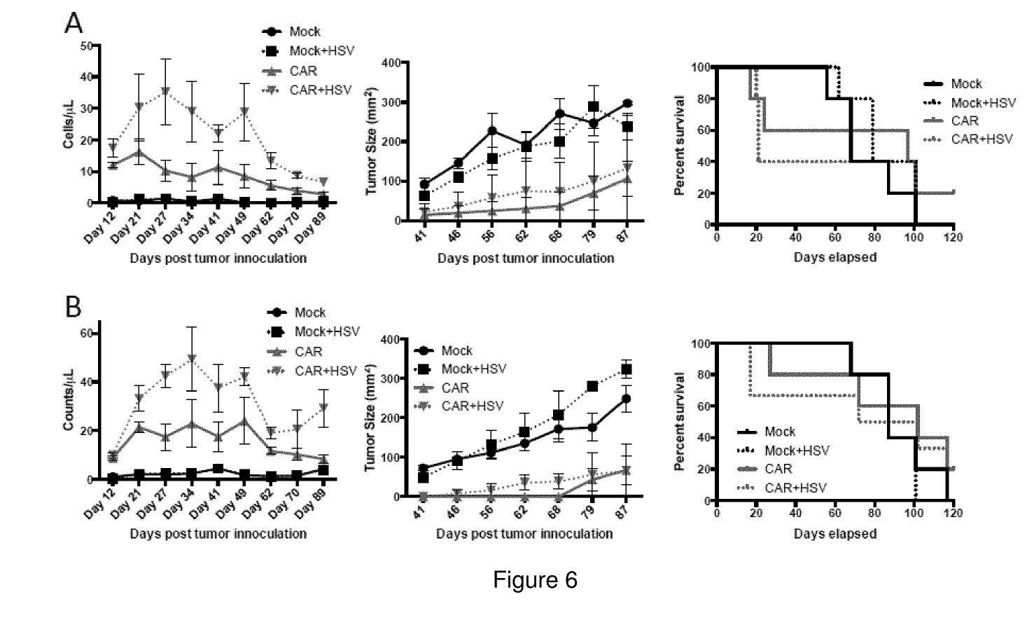

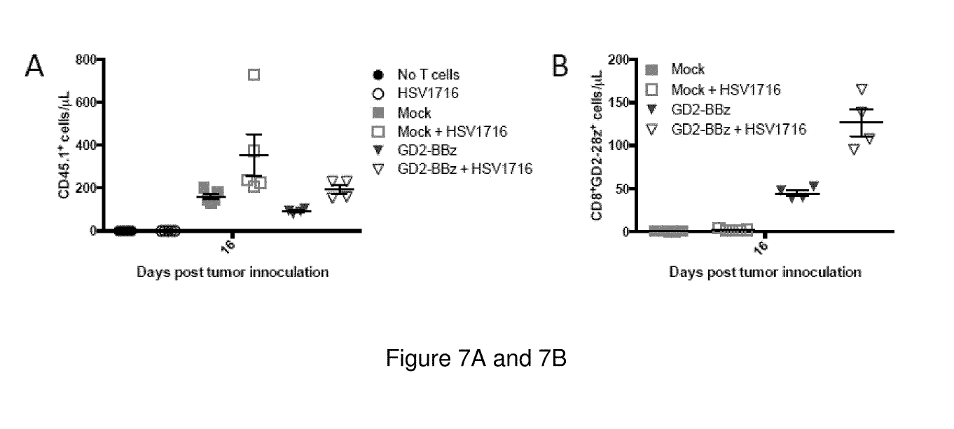

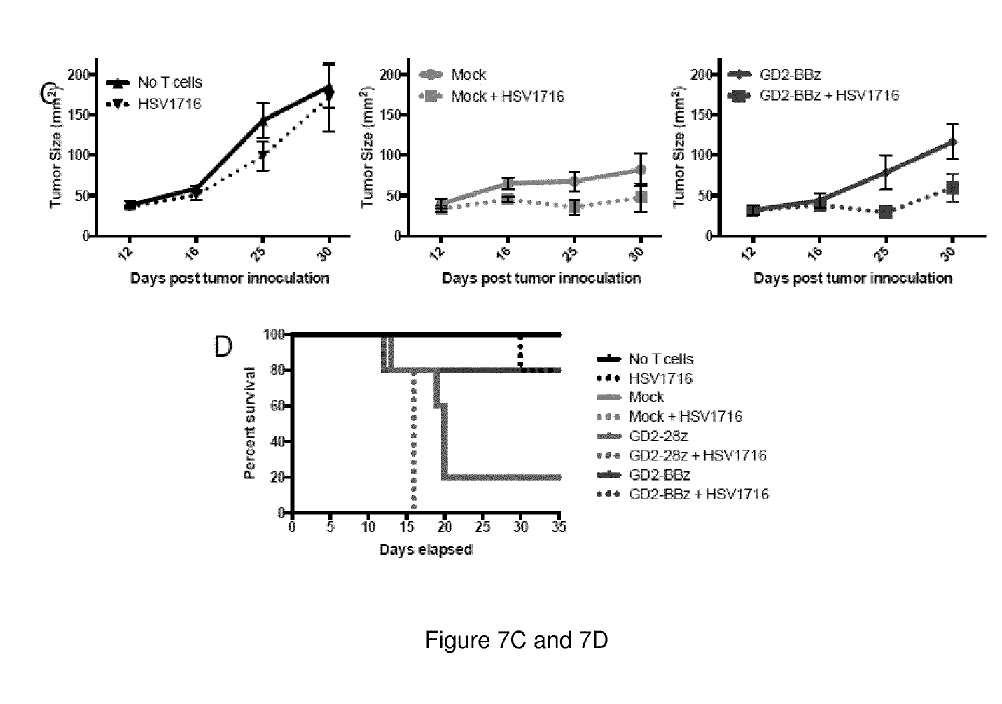

Results are shown in FIGS. 1A to 7D.

Our results showed that GD2 CAR T cells target GD2+ tumor cells in vitro and in vivo, and delay tumor growth. HSV1716 oncolytically lyses nectin 1-expressing cells, enhances GD2 CAR persistence in vivo, and delays tumor growth.

### Example 2—Oncolytic Virotherapy-Enhanced Chimeric Antigen Receptor T-Cell Therapy in Pediatric Solid Tumors

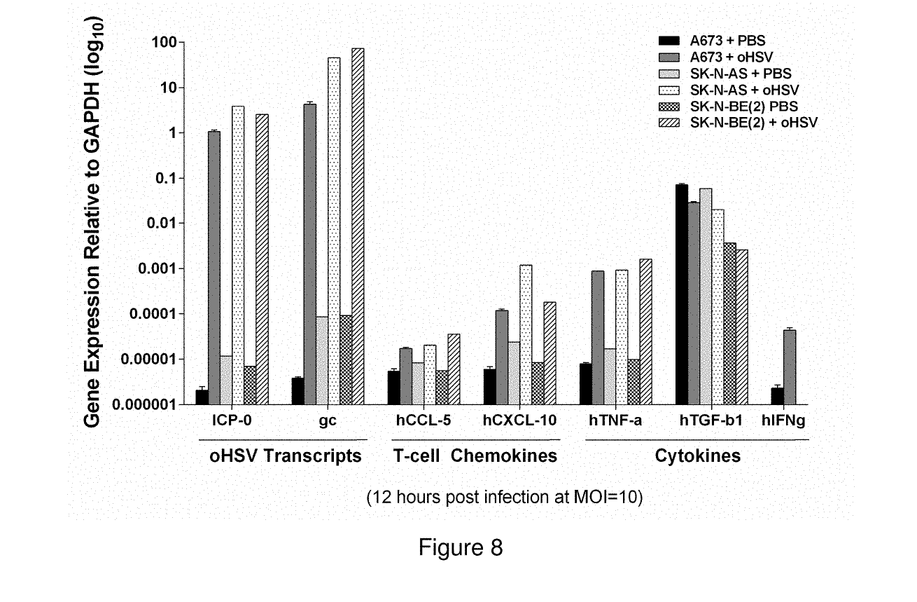

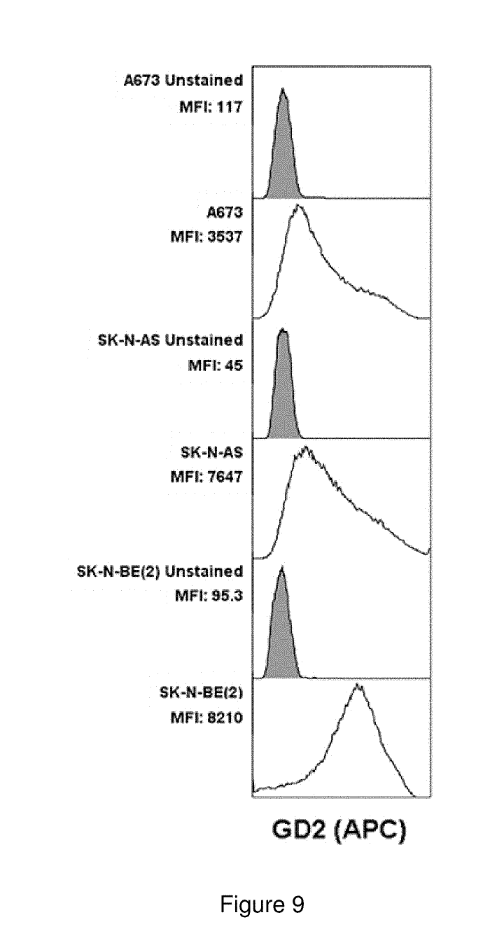

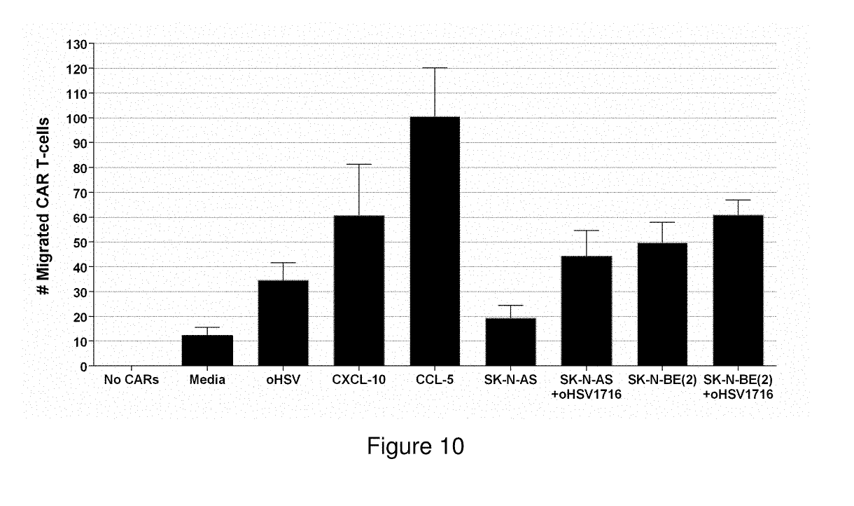

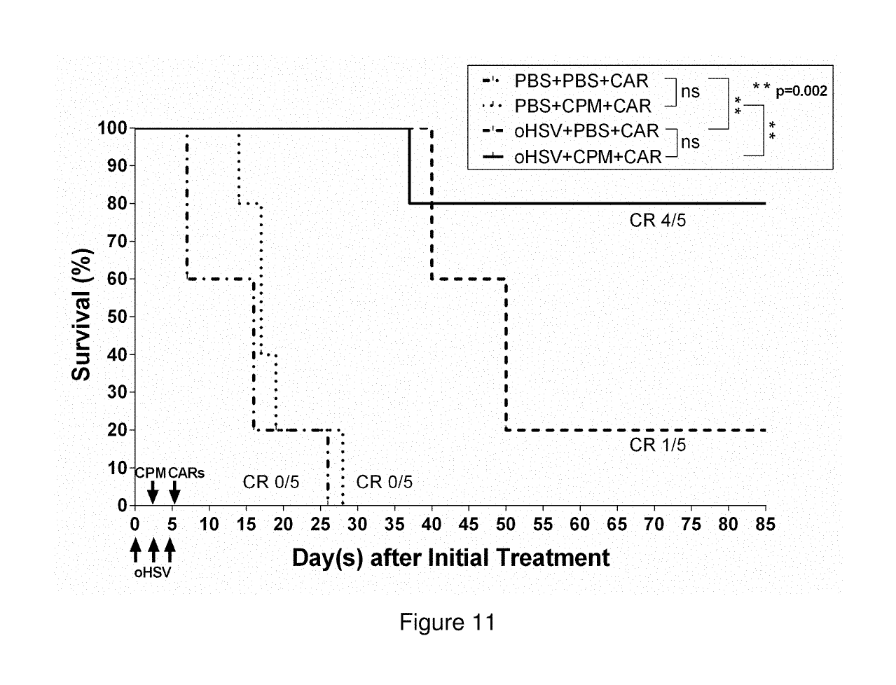

While chimeric antigen receptor (CAR) T-cell therapies have shown remarkable anticancer efficacy in patients with relapsed and refractory lymphoid leukemias, their effectiveness in patients with solid tumors has thus far been disappointing. Trials of treatment in solid tumors have shown little clinical success, with modest homing to tumors and lack of CAR persistence. These findings may be attributed to the immunosuppressive microenvironment characteristic of solid tumors. Oncolytic virotherapy is a promising platform which may potentiate the competence of CAR T-cells within solid tumors. Oncolytic viruses specifically amplify in malignant tissues and cause tumor-specific cell death not only through direct cell lysis, but also through the induction of an immunologic response. This mechanism suggests that oncolytic virotherapy may be a useful strategy to reverse the immune-escape tactics of solid tumors and augment the effects of directed T-cell therapies. We sought to determine whether the use of oncolytic Herpes Simplex virotherapy (oHSV) might enhance the efficacy of CAR T-cells in pediatric solid tumors. HSV1716 (trade name Seprehvir, Virttu Biologics, Ltd., Glasgow, U.K.) is a mutant Herpes Simplex-1 virus that lacks the RL1 gene encoding the virulence factor ICP34.5. This deletion nullifies the virus' ability to counteract host cell anti-viral responses and effectively restricts virus replication to cancer cells in which these mechanisms are absent or impaired. Seprehvir's safety has been demonstrated in multiple phase I clinical trials, including an ongoing trial for adolescents and young adults with refractory solid tumors initiated by our laboratory team (NCT00931931). We characterized the chemokine and cytokine profiles of human Ewing sarcoma and neuroblastoma cell lines before and after oHSV inoculation. We performed transwell migration assays of third-generation (containing CD28, OX40, and CD3z signaling domains) GD2-directed human CAR T-cells before and after the addition of Seprehvir in these models in vitro. We then performed in vivo survival studies using athymic nude mice and cyclophosphamide (CPM) lymphodepletion prior to CAR therapy. Our preliminary results suggest that infection of these pediatric solid tumor models with Seprehvir induces an immune response, which includes the T-cell attractant chemokines CXCL-10 (IP-10) and CCL-5 (RANTES) and T-cell activating cytokines such as IFN-γ and TNF-α while down-regulating such inhibitory cytokines as TGF-β (FIG. 8). Flow cytometry analysis revealed variable tumoral GD2 surface expression on each of these models (FIG. 9), while the CAR T-cells displayed high CXCR-3 and CCR-5 surface expression, allowing for chemotactic signaling through CXCL-10 and CCL-5, respectively (data not shown). These CAR T-cells displayed increased migration toward oHSV-infected tumor cells over non-infected cells (FIG. 10). Mice treated with combination therapy had significantly delayed tumor growth (data not shown) and prolonged survival when compared to CAR treatment alone, with 80% versus 0% of mice cured, respectively (FIG. 11). These results indicate that the addition of the oHSV construct Seprehvir is a valuable adjunct to GD2-directed CAR T-cell therapy in GD2-expressing pediatric solid tumors and should be further explored in clinical trials.

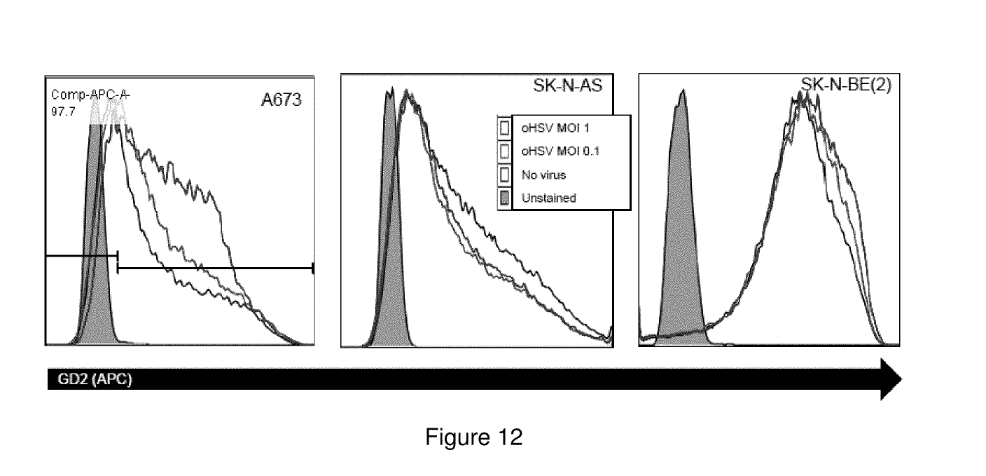

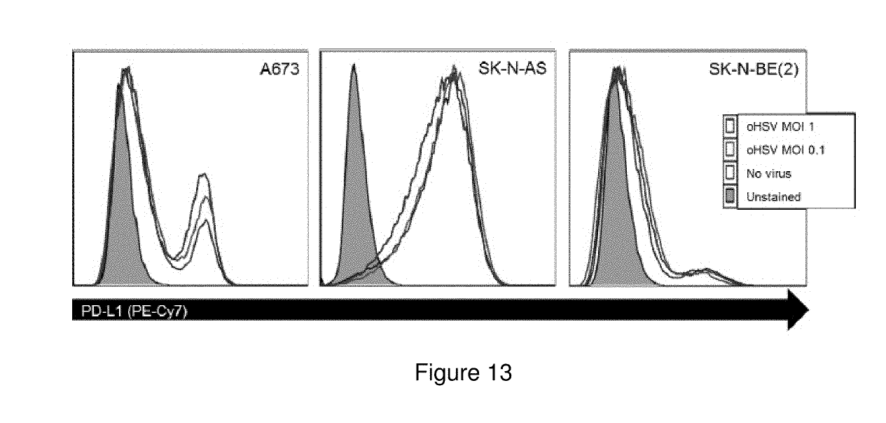

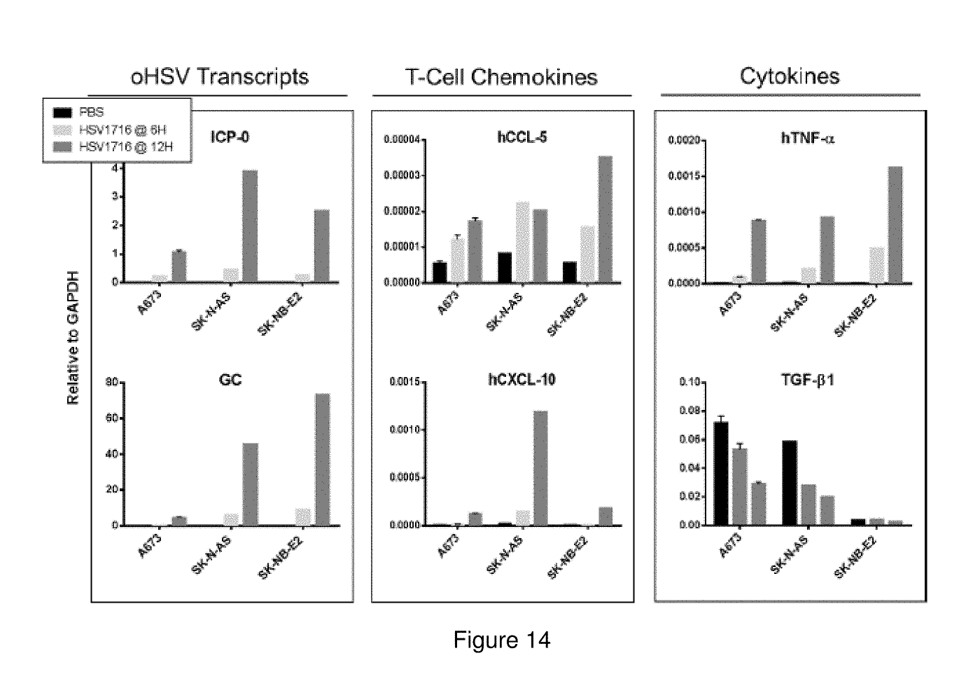

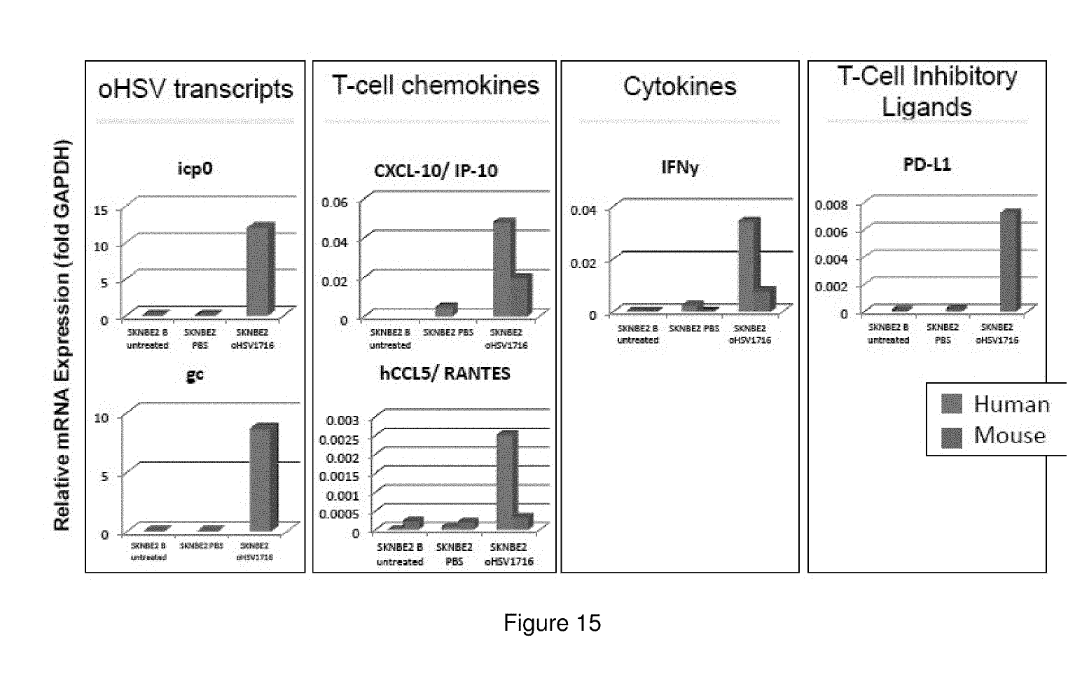

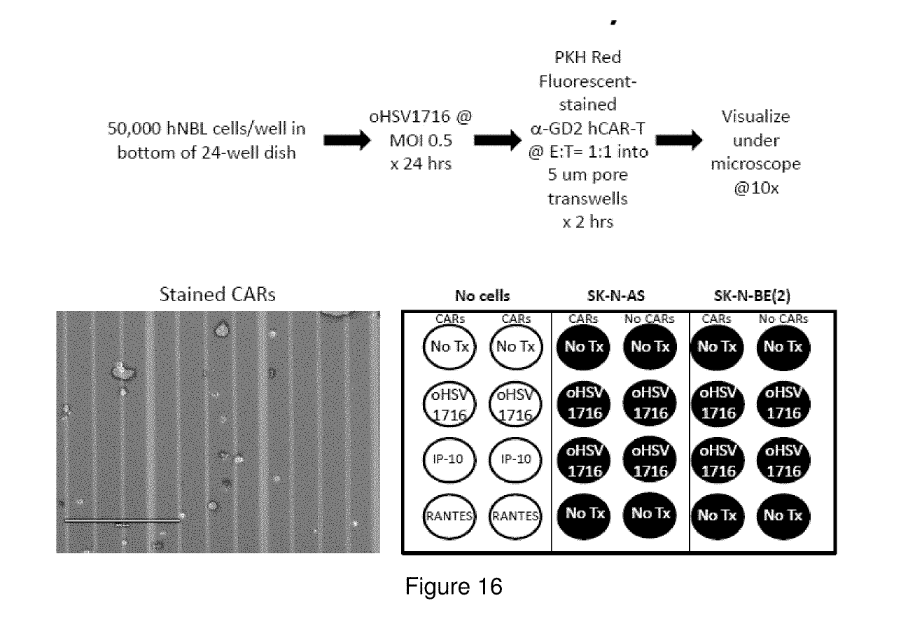

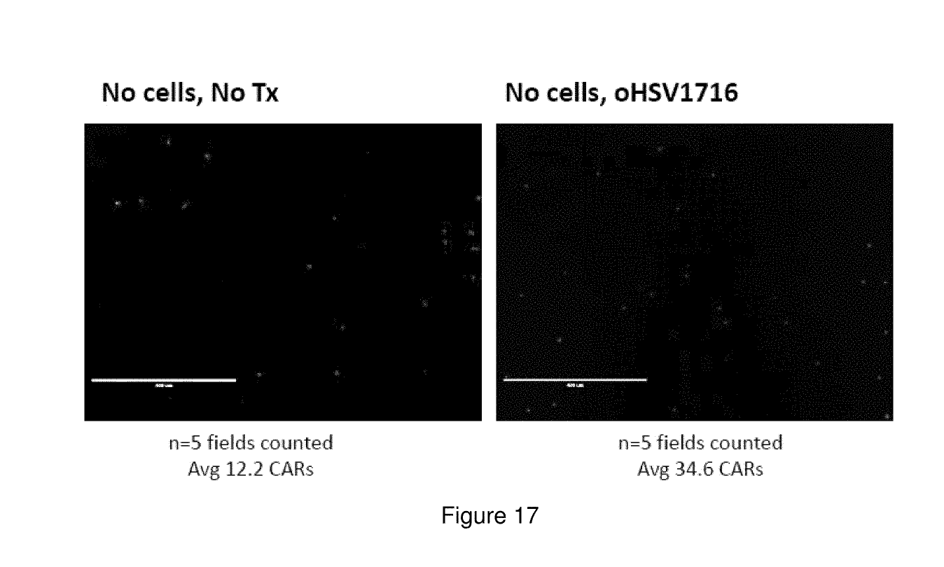

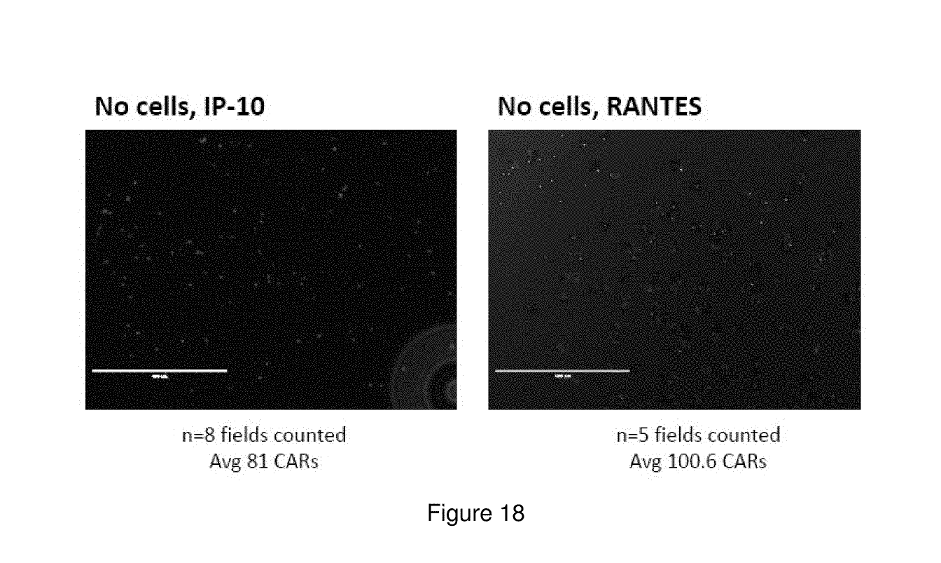

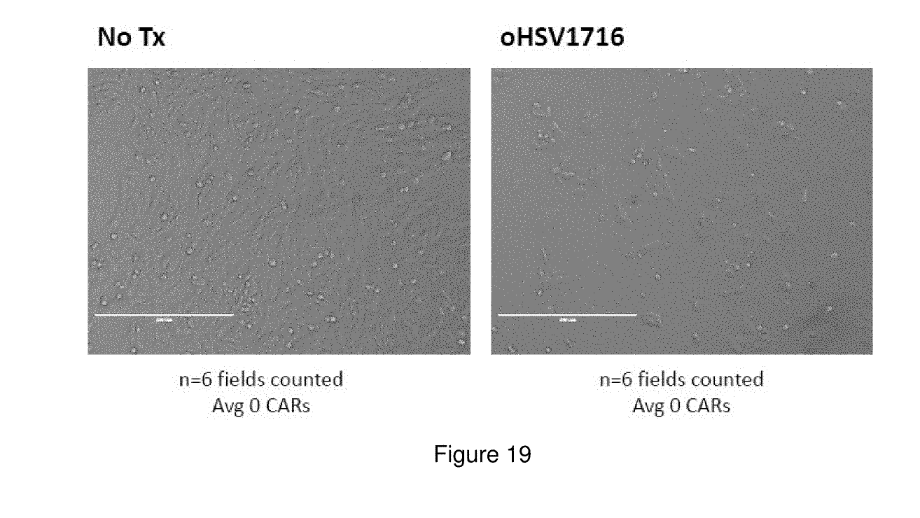

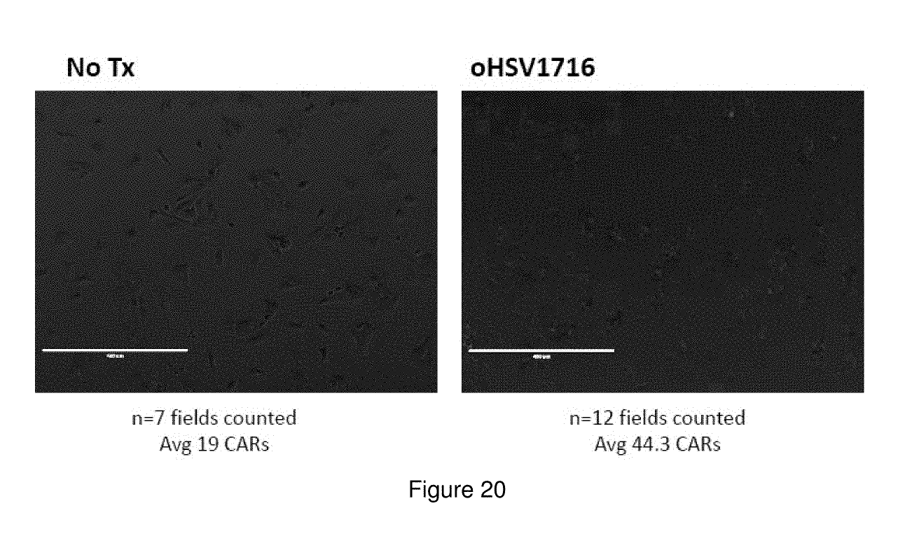

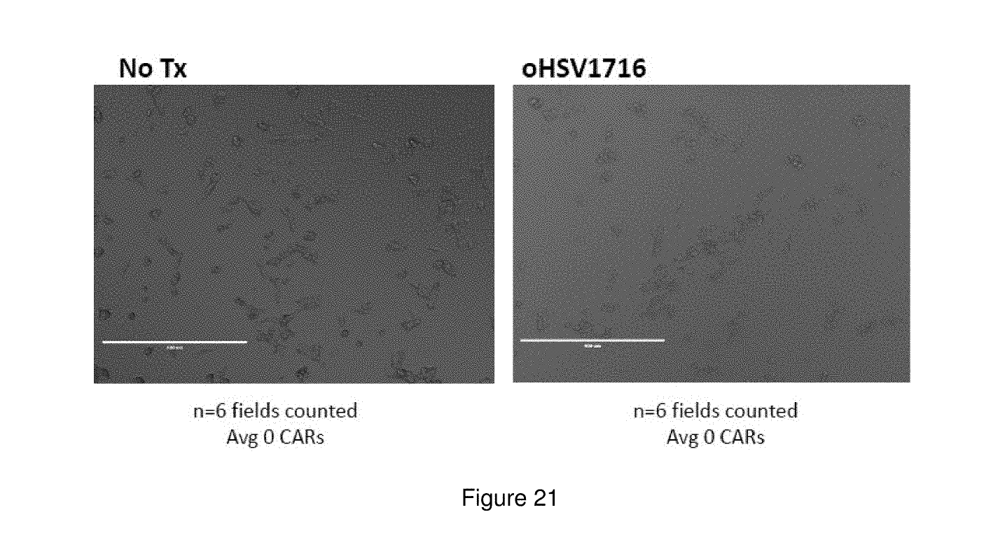

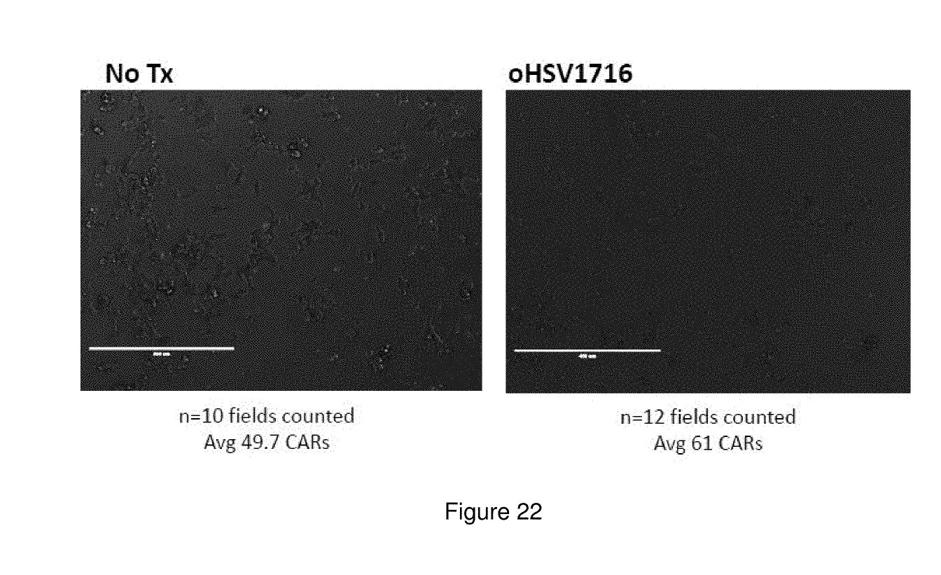

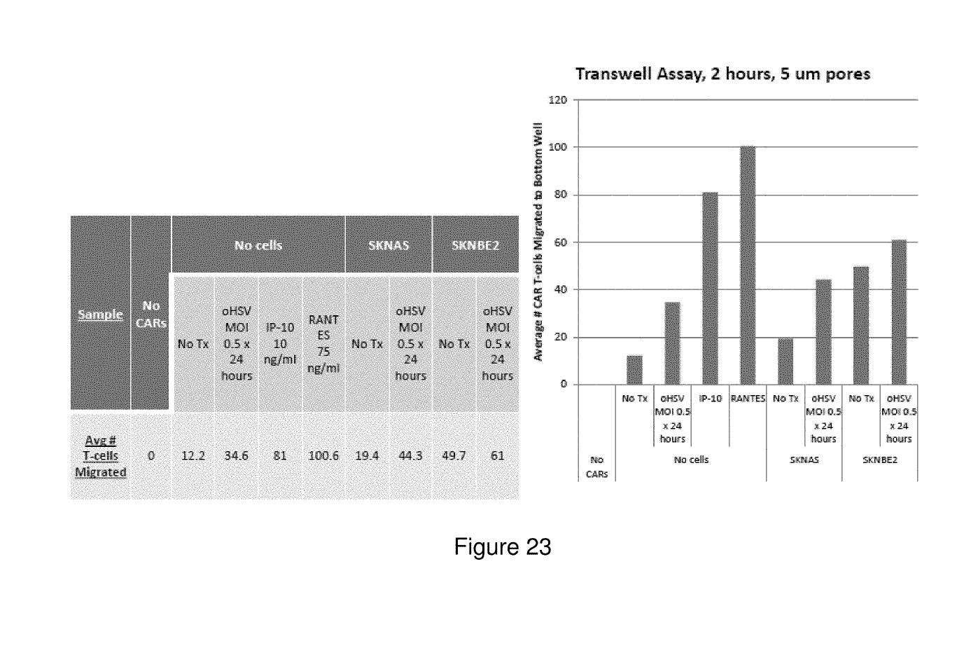

FIGS. 12 to 23 show results for experiments with α-GD2 hCAR-T cells +/−oHSV1716 for GD2-Positive Pediatric Solid Tumor Models In Vitro.

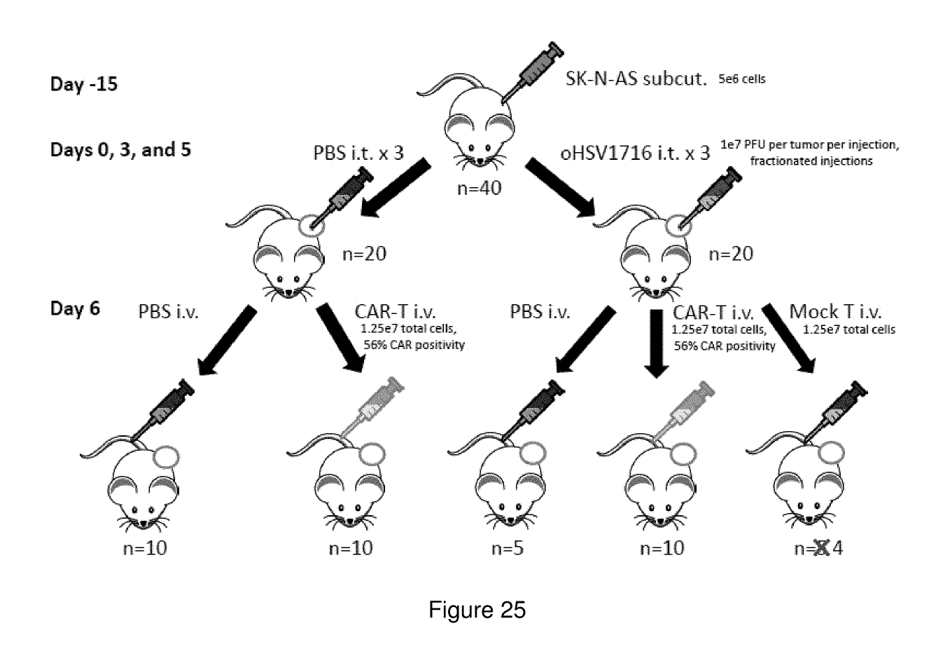

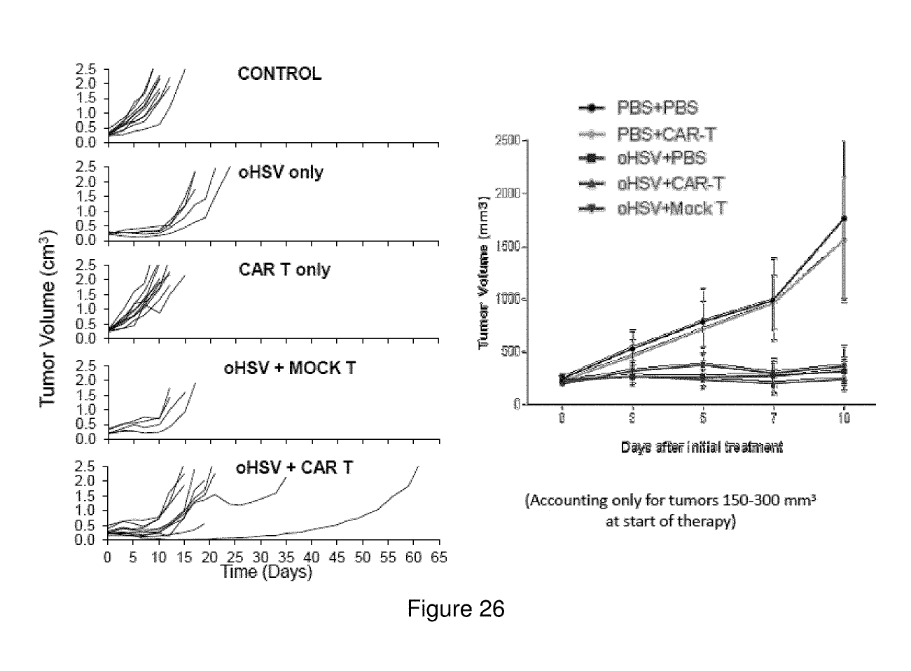

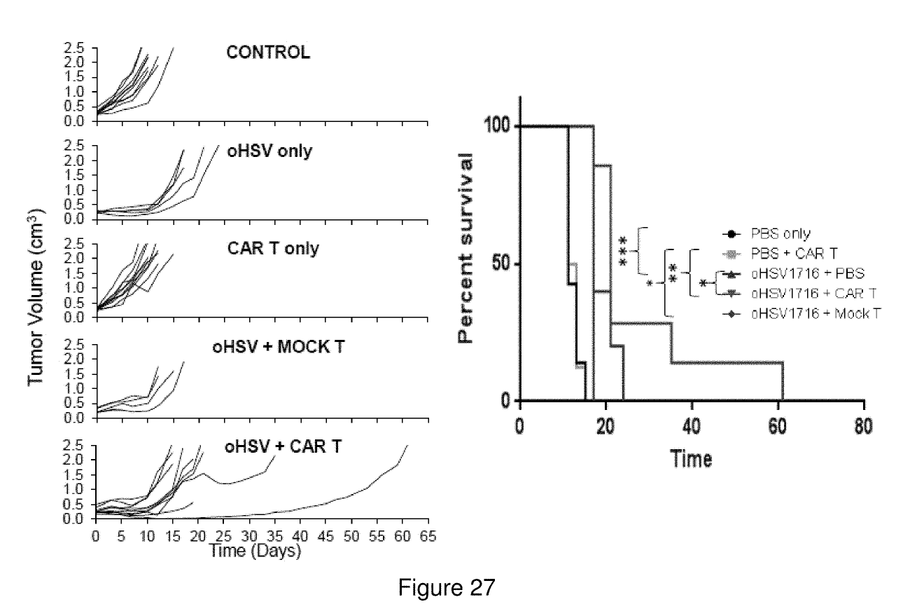

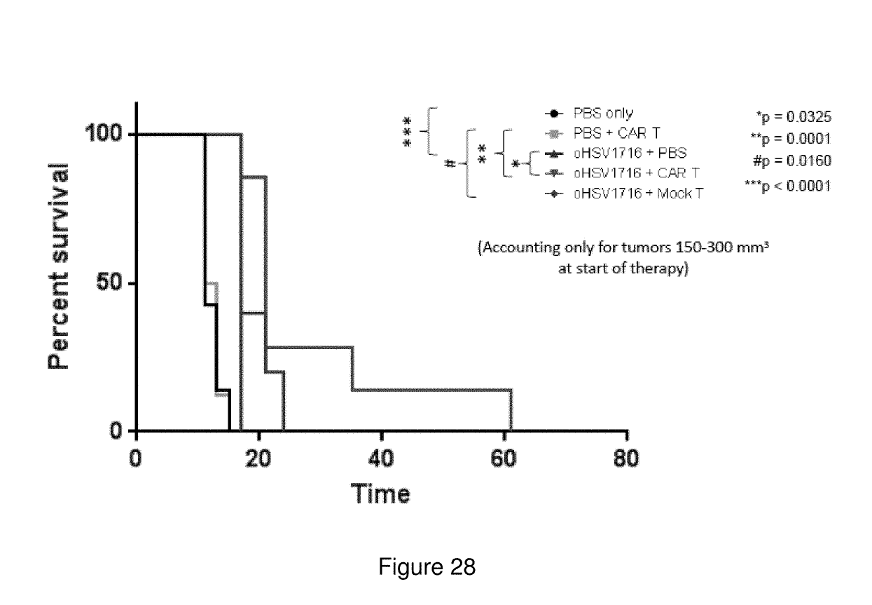

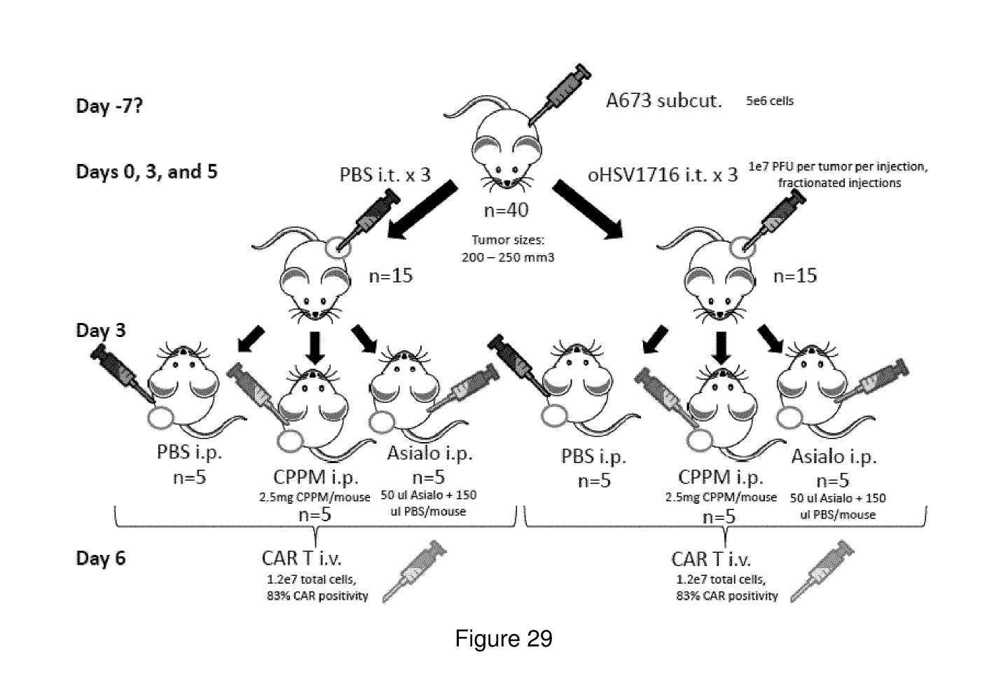

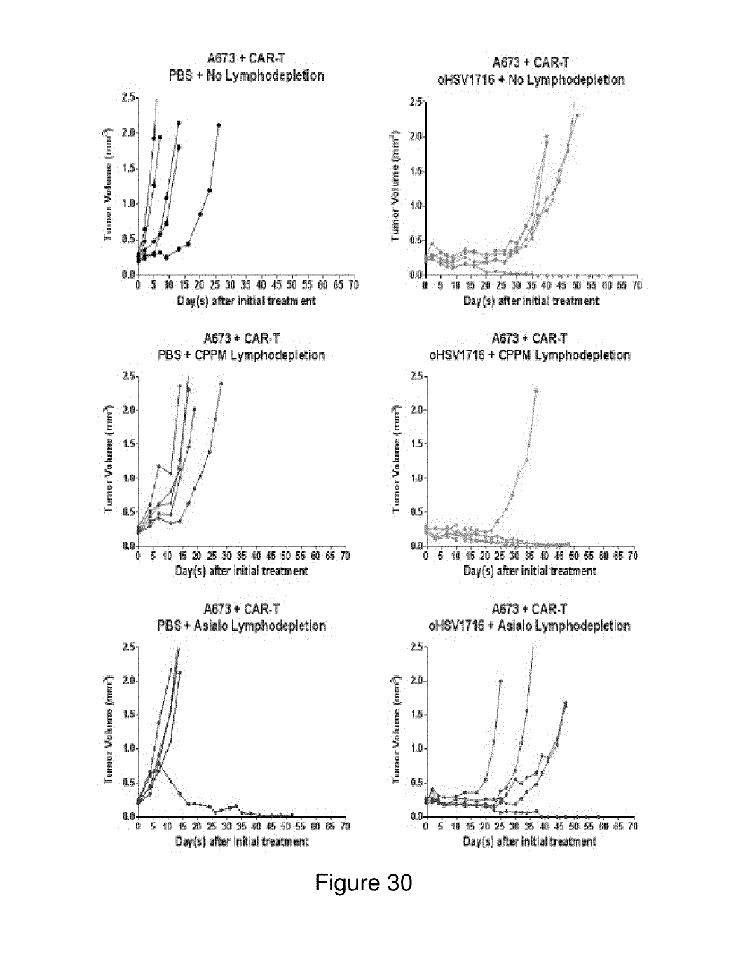

FIGS. 8 to 11 and 24 to 30 show results for experiments with α-GD2 hCAR-T cells +/−oHSV1716 for GD2-Positive Pediatric Solid Tumor Models In Vivo.

Experiment 1:

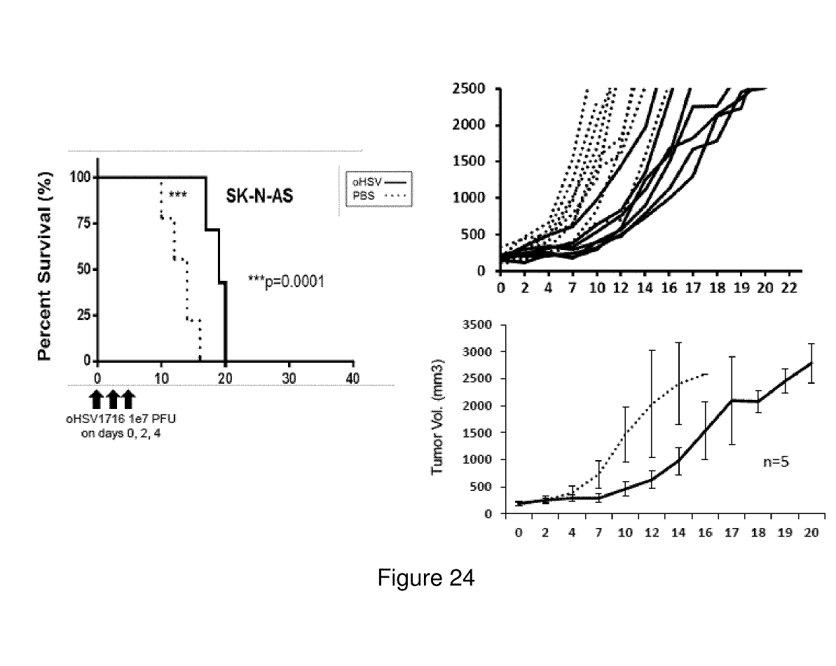

SK-N-AS In Vivo #1 Efficacy of Combination of oHSV1716+GD2 CAR T-cells in Pediatric Solid Tumor Xenograft Model SK-N-AS In Vivo (FIGS. 24 to 28).

SK-N-AS+/−oHSV+/−CAR-T study conclusions:
There is no significant difference between oHSV+PBS and oHSV+Mock-T survival curves
There is a significant survival advantage for oHSV+CAR-T compared to oHSV+PBS arm
There is a very significant survival advantage for oHSV+CAR-T compared to PBS+CAR-T arm
Lack of efficacy of PBS+CAR-T arm may be in part due to low GD2 expression of SK-N-AS
Lack of significant efficacy of T-cell arms overall may be in part due to inherent mouse NK cells getting rid of T-cells

Experiment 2:

A673 In Vivo Lymphodepletion Pilot #1 Efficacy of Combination of oHSV1716+GD2 CAR T-cells with and without Lymphodepletion in Pediatric Solid Tumor Xenograft Model A673 In Vivo (FIGS. 8 to 11 and 29 to 30).

A673+/−Lymphodepletion+/−oHSV1716+GD2 CAR T-cells study conclusions:
Lymphodepletion with CPPM in the absence of oHSV results in improvement of survival compared to no lymphodepletion or NK depletion with Asialo
Lymphodepletion did not seem to have a significant effect on tumor growth or mouse survival when combined with oHSV
All oHSV arms have superior survival benefit compared to PBS arms

### Example 3—Oncolytic Virotherapy Enhances GD2-Directed Chimeric Antigen Receptor (CAR) T-Cell Therapy in GD2-Expressing Pediatric Solid Tumor Xenograft Models

High Risk Neuroblastoma (NBL) is the most common non-CNS pediatric solid tumor, requires multimodal and targeted therapy, is responsible for ˜15% total childhood cancer deaths and has <10% survival for ˜50% of children who relapse

Ewing Sarcoma (EWS) is among most prevalent solid tumor afflicting older children and adolescents, ˜30% are refractory to conventional therapy, there is ˜30% survival for patients with metastases.

GD2 is a disialoganglioside expressed on NBL and EWS, and is a strategic immunotherapeutic target.

Chimeric Antigen Receptor (CAR) T-Cells are engineered T-cells targeted against tumor antigen, have remarkable efficacy in relapsed/refractory lymphoid leukemias. CAR T cells have so far shown little clinical success against solid tumors, modest migration to tumor, lack of activation, proliferation, and persistence. These limitations are attributable to the solid tumor immunosuppressive microenvironment.

Oncolytic Herpes Simplex Virotherapy (oHSV) is tumor selective, subject of recent FDA approval with several open clinical trials. It combines two antitumor efficacy mechanisms: a direct lytic effect and induction of immune response.

While chimeric antigen receptor (CAR) T-cell therapies have shown remarkable anticancer efficacy in patients with relapsed and refractory lymphoid leukemias, their effectiveness in patients with solid tumors has been more challenging. Among the barriers thought to interfere with CAR T cell efficacy are impaired homing to tumors and poor CAR T cell persistence, likely attributable to the immunosuppressive microenvironment. Due to their pro-inflammatory effects, oncolytic viruses are strong candidates to potentiate the competence of CAR T cells within solid tumors. Seprehvir (HSV1716) is an HSV-1 attenuated by deletion of the RL1 gene encoding the neurovirulence protein ICP34.5. The virus has a long track record of safety in clinical trials and is currently being tested in adolescents and young adults with refractory solid tumors (NCT00931931, NCT02031965). We hypothesized that intra-tumoral administration of Seprehvir enhances GD2-directed CAR T cell efficacy. We characterized the chemokine and cytokine profiles of human GD2-positive Ewing sarcoma and neuroblastoma cell lines before and after oHSV inoculation. We performed transwell migration assays of third-generation (containing CD28, OX40, and CD3z signaling domains) GD2-directed human CAR T-cells before and after the addition of Seprehvir in these models in vitro. We then performed in vivo survival studies using athymic nude mice and cyclophosphamide (CPM) lymphodepletion prior to CAR therapy. Our results suggest that infection of these pediatric solid tumor models with Seprehvir induces an immune response, which includes the T-cell attractant chemokines CXCL-10 (IP-10) and CCL-5 (RANTES) and T-cell activating cytokines such as IFN-g and TNF-α, while down-regulating such inhibitory cytokines as TGF-b. Flow cytometry analysis revealed variable tumoral GD2 surface expression on each of these models, while the CAR T-cells displayed high CXCR-3 and CCR-5 surface expression, allowing for chemotactic signaling through CXCL-10 and CCL-5, respectively. The CAR T-cells displayed increased migration toward oHSV-infected tumor cells over non-infected cells. Mice treated with combination therapy had significantly delayed tumor growth and prolonged survival when compared to CAR treatment alone. Despite being athymic nude mice, the majority of mice cured by combination therapy were resistant to tumor re-challenge, suggesting the long-term persistence of CAR T cells. These results indicate that the addition of Seprehvir may be a valuable adjunct to CAR T-cell therapy and should be further explored in clinical trials.

In vitro, we:
Characterized oHSV-induced chemokine/cytokine gene expression by RT-PCR
   Tumor cells cultured with Seprehvir at multiplicity of infection (MOI)=10×12 hours
Determined tumoral GD2 expression by flow cytometry
Determined CAR T-cell CXCR-3 and CCR-5 expression by flow cytometry
Performed transwell migration assays:
   Tumor cells cultured with Seprehvir at MOI=1×24 hours
   Red fluorescent PKH23-stained CAR T-cells added to 5 mm pore inserts×2 hours
   Negative control: media alone
   Positive controls: media with 75 ng/ml CXCL-10 (IP-10) or 10 ng/ml CCL-5 (RANTES)
   Cells quantified through microscopic visualization
   Results represent averages of n replicates for each sample

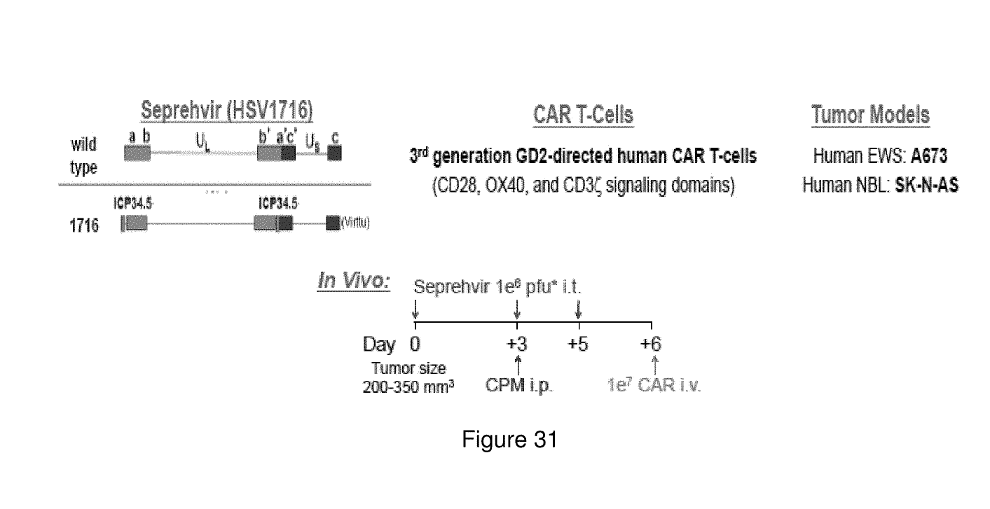

In vivo:
Athymic nude mice with subcutaneous flank tumors
PBS or Seprehvir was administered intra-tumorally (i.t)×3 (FIG. 31)
Intra-peritoneal (i.p.) PBS or cyclophosphamide (CPM)×1 prior to CAR treatment
Intravenous (i.v.) PBS or CAR T-cells×1

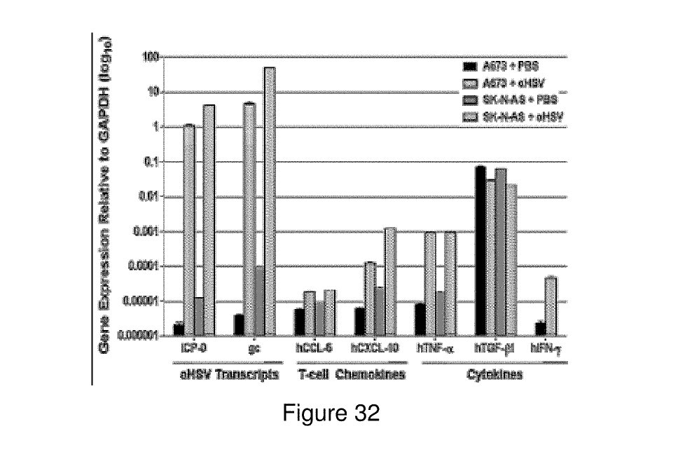

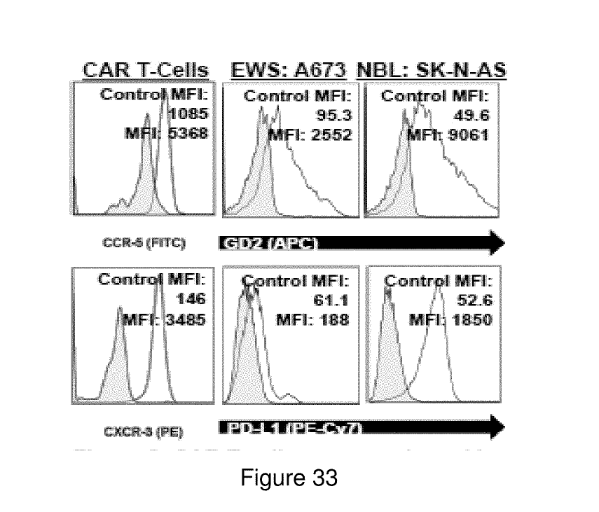

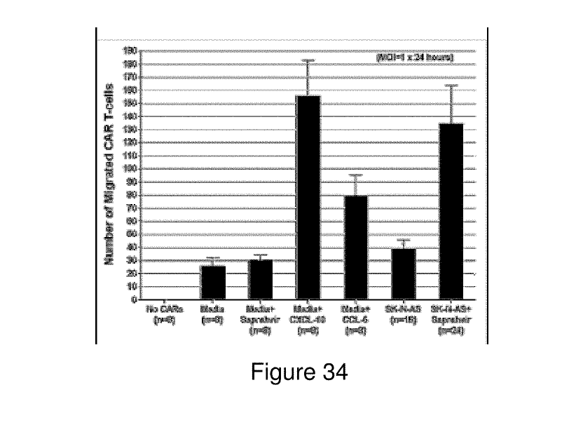

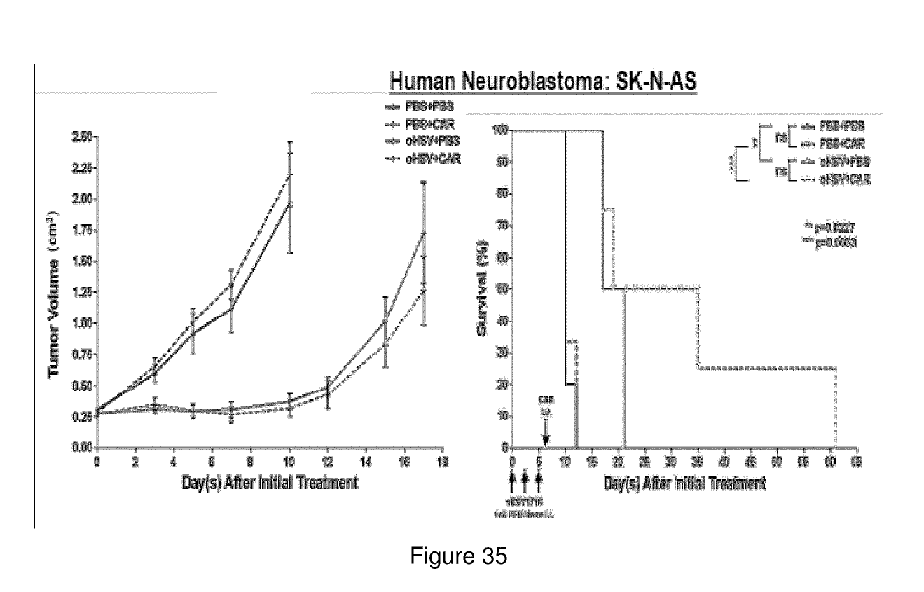

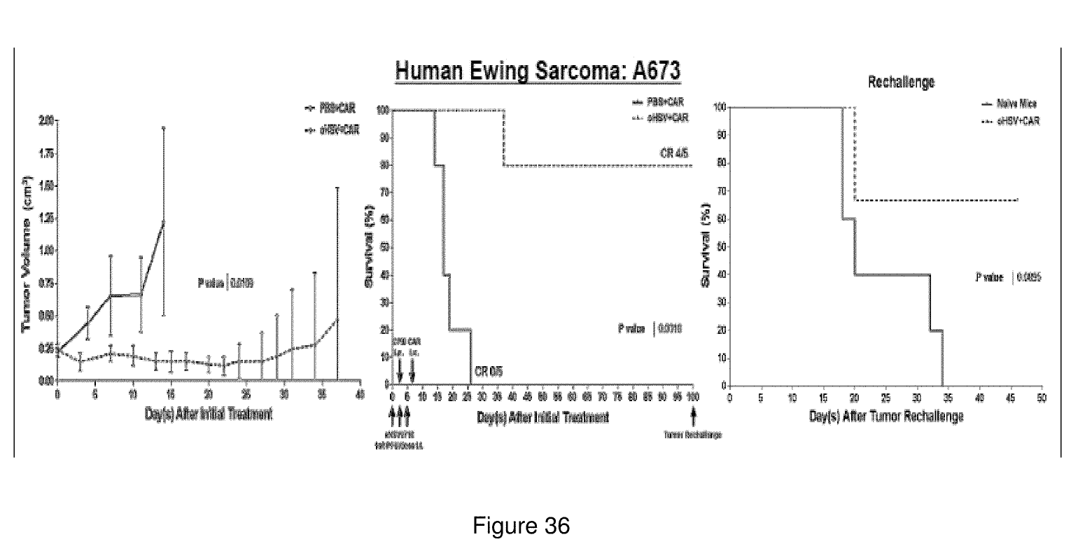

Results are shown in FIGS. 31 to 36.

Our results showed that oHSV infection induces release of chemokines and cytokines that promote CAR T-cell migration and activation; oHSV enhances GD2-directed human CAR T-cell antitumor efficacy against GD2-expressing pediatric solid tumors. oHSV is a promising adjunct to CAR T-cell therapy for pediatric solid tumors.

### REFERENCES

1. Park, J. R., et al. Children's Oncology Group's 2013 blueprint for research: neuroblastoma. Pediatric blood & cancer. 6: 985-993. (2013).
2. Gorlick, R., et al. Children's Oncology Group's 2013 blueprint for research: bone tumors. Pediatric blood & cancer. 6: 1009-1015. (2013).
3. Lipinski, M., et al. Neuroectoderm-associated antigens on Ewing's sarcoma cell lines. Cancer research. 1: 183-187. (1987).
4. Yu, A. L., et al. Anti-GD2 antibody with GM-CSF, interleukin-2, and isotretinoin for neuroblastoma. The New England journal of medicine. 14: 1324-1334. (2010).
5. Maude, S. L., et al. Chimeric antigen receptor T cells for sustained remissions in leukemia. The New England journal of medicine. 16: 1507-1517. (2014).
6. Singh, N., et al. Nature of tumor control by permanently and transiently modified GD2 chimeric antigen receptor T cells in xenograft models of neuroblastoma. Cancer immunology research. 11: 1059-1070. (2014).
7. Chen, D. S. & Mellman, I. Oncology meets immunology: the cancer-immunity cycle. Immunity. 1: 1-10. (2013).
8. Dolan, A., McKie, E., MacLean, A. R. & McGeoch, D. J. Status of the ICP34.5 gene in herpes simplex virus type 1 strain 17. The Journal of general virology. 971-973. (1992).
9. Workenhe, S. T., Verschoor, M. L. & Mossman, K. L. The role of oncolytic virus immunotherapies to subvert cancer immune evasion. Future oncology. 4: 675-689. (2015).
10. Nishio, N., et al. Armed oncolytic virus enhances immune functions of chimeric antigen receptor-modified T cells in solid tumors. Cancer research. 18: 5195-5205. (2014).
11. NCT00931931. HSV1716 in Patients With Non-Central Nervous System (Non-CNS) Solid Tumors. 2009; January 9.
12. Cassady, K. A., et al., To Infection and Beyond: The Multi-Pronged Anti-Cancer Mechanisms of Oncolytic Viruses. Viruses, 2016. 8(2).

1. A method of treating cancer in a subject, the method comprising administration of an oncolytic herpes simplex virus and administration of human lymphocyte cells modified to express a chimeric antigen receptor (CAR) or modified to express a T cell receptor (TCR).

2. The method of claim 1, wherein the lymphocyte cells are T-cells.

3. The method of claim 1, wherein the T-cells are cytotoxic T-cells, CD8+ T cells or CD4+ T cells.

4. The method of claim 1, wherein the cancer is a solid tumor.

5. The method of claim 1, wherein the CAR or TCR targets an antigen selected from the group consisting of GD2, CD44v7/8, DNAM-1 (DNAX accessory molecule-1), EGP-40 (epithelial glycoprotein-40), EpCAM (endothelial cell adhesion molecule), FBP (folate-binding protein), FR, GD3, VEGFR2, LMP-1 (latent membrane protein 1), MUC1 (mucin 1), PSCA (prostate stem cell antigen), α-folate receptor, CD171, CAIX, Her2, IL13Rα2, IL13R, IL3RA, CEA, CD19, CD20, Lewis-Y, CD33, CD38 (also known as cyclic ADP ribose hydrolase), CD123, gp100, MART1, CEA, CAIX, Her2//Neu, MAGE-A3/A19/A12, MAGE-A3/titin, CD19, GD2, NY-ESO-1, CTAG1B, MAGE-A1, MAGE-C1, SSX2, MAGE-A2B, Brachyury, NY-BR1, BCMA, KRAS (e.g. KRAS G13D, KRAS G12V, KRAS G12R, KRAS G12D, KRAS G12C), KIT, PD-L1, EGFRviii, HPV 16 E6, HPV 16 E7, HPV18 E6, HPV18 E7 and other tumor associated antigens

6. The method of claim 1, wherein the administration of the oncolytic herpes simplex virus and lymphocyte cells is simultaneous or sequential.

7. The method of claim 1, wherein the oncolytic herpes simplex virus is administered to the blood.

8. The method of claim 1, wherein the oncolytic herpes simplex virus is administered by intratumoral injection.

9. The method of claim 1, wherein the administration of human lymphocyte cells is part of a method of autologous therapy.

10. The method of claim 1, wherein the oncolytic herpes simplex virus does not express, or is not modified to express, a cytokine or chemokine.

11. The method of claim 1, wherein the oncolytic herpes simplex virus does not contain, or is not modified to contain, nucleic acid encoding at least one copy of a polypeptide that is heterologous to the virus.

12. The method of claim 1, wherein the oncolytic herpes simplex virus is an HSV-1 strain 17+ or mutant thereof.

13. The method of claim 1, wherein the oncolytic HSV is HSV1716.

14. A method of increasing the efficacy of adoptive cell therapy in a subject by administering an oncolytic herpes simplex virus to a subject in need thereof.

15. A kit comprising at least one container having a predetermined quantity of oncolytic herpes simplex virus, and at least one container having a predetermined quantity of human lymphocytes modified to express a chimeric antigen receptor (CAR) or T cell receptor (TCR).

16. The kit of claim 15, wherein the oncolytic herpes simplex virus and lymphocytes are in separate containers.

17. The kit of claim 15, wherein the kit comprises a container having a mixture of a predetermined quantity of oncolytic herpes simplex virus and predetermined quantity of human lymphocytes.
